(19) United States
(12) Patent Application Publication
(10) Pub. No.: US 2022/0220202 A1
(43) Pub. Date: Jul. 14, 2022

(54) COMBINATION THERAPY FOR TREATMENT AND PREVENTION OF AUTOIMMUNE AND INFLAMMATORY DISEASES

(71) Applicant: MONASH UNIVERSITY, Clayton, Victoria (AU)

(72) Inventors: CHARLES REAY MACKAY, Clayton, Victoria (AU); REMY ROBERT, Clayton, Victoria (AU); ELIANA MARINO MORENO, Clayton, Victoria (AU)

(73) Assignee: MONASH UNIVERSITY, Clayton, Victoria (AU)

(21) Appl. No.: 17/280,170

(22) PCT Filed: Oct. 9, 2019
(86) PCT No.: PCT/AU2019/051090

§ 371 Date: Mar. 25, 2021

(30) Foreign Application Priority Data
Oct. 9, 2018 (AU) 2018903815

(51) Int. Cl. C07K 16/28; A23L 33/12; A61K 31/718; A61K 45/06

(52) U.S. Cl. CPC C07K 16/2809; A23L 33/12; A61K 2039/505; A61K 45/06; A61K 31/718

(57) ABSTRACT

The invention relates to the combination and delivery of compounds for the treatment and prevention of autoimmune and inflammatory diseases. Further, the invention relates to methods of treating a condition associated with effector and/or memory T cells, or other pathogenic immune cells the method comprising, consisting essentially of or consisting of reducing the activity of, or number of, pathogenic immune cells (preferably T cells) in an individual, and providing in the individual, a therapeutically effective amount of at least one type of short chain fatty acid, ester or salt thereof, thereby treating the condition associated with effector and/or memory T cells or other pathogenic immune cells. The present invention also provides compositions for use in methods of the invention.

### CROSS REFERENCE TO RELATED APPLICATIONS

The present application is a § 371 National Phase Application of PCT/AU2019/051090, filed Oct. 9, 2019, which application claims priority to Australian Patent Application No. 2018903815, filed Oct. 9, 2018, the entire contents of which are hereby incorporated in their entirety for all purposes.

### SEQUENCE LISTING

The instant application contains a Sequence Listing which has been submitted electronically in ASCII format and is hereby incorporated by reference in its entirety. Said ASCII copy, created on May 12, 2021, is named 53050206AZD_ST25.txt and is 8.75 KB in size.

### FIELD OF THE INVENTION

The invention relates to the combination and delivery of compounds for the treatment and prevention of autoimmune and inflammatory diseases.

### RELATED APPLICATION

This application claims priority from Australian provisional application AU 2018903815, the contents of which are hereby incorporated by reference in their entirety.

### BACKGROUND OF THE INVENTION

The ability to distinguish foreign microorganisms from host cells is crucial to avoid detrimental localised damage to self during an inflammatory response. However, there are chronic diseases where immune cells begin to target and damage host cells and tissues. When adaptive cells break self-tolerance, the resulting condition is characterised as autoimmunity, where native host cells are recognised as foreign and the adaptive immune cells target them for destruction.

Autoimmune diseases cause significant human morbidity and mortality. These diseases include approximately 80 diseases, such as rheumatoid arthritis, systemic lupus, systemic lupus erythematosus (SLE), and multiple sclerosis, and affect approximately 5% of the population of the United States. Dysregulation of the immune system plays a critical underpinning role in conditions such as autoimmune diseases.

The burden of autoimmune disease is significant, with a substantial minority of the western population (2-5%) suffering from this group of diseases. Women are also more susceptible to autoimmune disease, particularly in child-bearing years, such that autoimmune disease is estimated as being among the leading causes of death of women in the US in all age groups up to 65.

There are no cures for autoimmune disease, and current therapies are typically aimed at managing the pain associated with the disease (for example, using steroids or non-steroidal anti-inflammatories) or at reducing the inflammatory response using immunosuppressants. Immunosuppressive pharmaceuticals can be prohibitively expensive, reducing access to therapy for many sufferers. In the case of autoimmune disease which results in the destruction of functional cells (for example, type 1 diabetes, in which pancreatic beta cells are destroyed), there are even more limited treatment options, with exogenous insulin treatment remaining the primary treatment approach.

In addition to classical autoimmune diseases, a number of inflammatory conditions represent a significant health burden. These include inflammatory bowel diseases (Crohn's disease, Ulcerative colitis) and fatty liver disease (non-alcoholic steato-hepatitis). In such diseases, no autoantigen has yet been defined, although the inflammatory process is considered important for driving disease.

A need exists for new and/or improved methods and compositions for treating and preventing autoimmune diseases and/or inflammatory diseases.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect the present invention provides a method for treating a condition associated with pathogenic immune cells, the method comprising, consisting essentially of or consisting of:
reducing the activity of, or number of, pathogenic immune cells in an individual; and
providing in the individual, a therapeutically effective amount of at least one type of short chain fatty acid, ester or salt thereof

thereby treating the condition associated with pathogenic immune cells.

In one aspect the present invention provides a method for treating a condition associated with effector and/or memory T cells, the method comprising, consisting essentially of or consisting of:
reducing the activity of, or number of, T cells in an individual; and
providing in the individual, a therapeutically effective amount of at least one type of short chain fatty acid, ester or salt thereof

thereby treating the condition associated with effector and/or memory T cells.

In another aspect the present invention provides a method for treating a condition associated with dysfunctional/ineffective regulatory T cell function, the method comprising, consisting essentially of or consisting of:
reducing the activity of, or number of, T cells in an individual; and
providing in the individual, a therapeutically effective amount of at least one type of short chain fatty acids, esters or salts thereof,
thereby treating the condition associated with dysfunctional/ineffective regulatory T cell function.

In another aspect the present invention provides a method for treating a condition associated with autoreactive T effector cells, the method comprising, consisting essentially of or consisting of:
reducing the activity of, or number of, T cells in an individual; and
providing in the individual, a therapeutically effective amount of at least one type of short chain fatty acid, ester or salt thereof,

thereby treating the condition associated with autoreactive T effector cells.

In any aspect, the reduction in the activity of T cells is a reduction in the activity of effector and/or memory T cells. Preferably, the reduction in the activity of T cells is a reduction in the activity of Th1, Th17 and/or Th22 cells.

In any aspect, the pathogenic immune cells that may be reduced in activity or number include B cells, subpopulations of NK cells, dendritic cells, all of which play a role in the pathogenesis of autoimmune diseases.

In any aspect, the reduction in the number of T cells is a reduction in the number of effector and/or memory T cells. Preferably, the reduction in the number of T cells is a reduction in the number of Th1, Th17 and/or Th22 cells.

In any aspect, the method or condition is associated with, caused by or is the result of the presence of autoreactive and/or pathogenic T cells and the method comprises reducing the activity or number of autoreactive and/or pathogenic T cells in the individual.

In any aspect, the method or condition is associated with, caused by or is the result of the presence of pathogenic immune cells and the method comprises reducing the activity or number of pathogenic immune cells in the individual.

In any aspect, the reduction in the activity or number of T cells, is a reduction in the activity or number of activated effector T cells, preferably with a lesser effect on regulatory T cells (Tregs).

In any aspect, the reduction in the activity or number of T cells, is a reduction in the activity or number of activated effector T cells, with little or no effect on regulatory T cells (Tregs).

In any aspect of the present invention, the step of reducing the activity of, or number of, effector and/or memory T cells in an individual comprises, consists essentially of or consists of administering to the individual an agent capable of reducing the viability of a T cell. The agent may reduce the viability of the T cell either directly or indirectly (i.e. mediate a reduction). Preferably, the agent is capable of depleting a T cell, or population of T cells, when administered to the individual.

In any aspect of the present invention, the step of reducing the activity of, or number of, pathogenic immune cells in an individual comprises, consists essentially of or consists of administering to the individual an agent capable of reducing the viability of an immune cell. Preferably, the immune cell is a T cell, a B cell, an NK cell or a dendritic cells. The agent may reduce the viability of the pathogenic immune cell either directly or indirectly (i.e. mediate a reduction). Preferably, the agent is capable of depleting a pathogenic immune cell, or population of pathogenic immune cells, when administered to the individual.

In any aspect of the present invention, a T cell may be any T cell. Preferably, the T cell is a Th1, Th17 or Th22 cell. Preferably, the T cell is a T cell that expresses CD3, CXCR3 and/or CCR6. Accordingly, a step in a method of the invention to reduce the activity of, or number of, T cells is a step of reducing the activity of, or number of, CD3+, CXCR3+ and/or CCR6+ T cells.

In any aspect of the present invention, reducing the number of pathogenic immune cells, preferably T cells, in an individual occurs by administering an antibody that binds to a pathogenic immune cell (such as a T cell) and either directly or indirectly results in a reduction in viability of the pathogenic immune cell. In one example, upon binding to the pathogenic immune cell (preferably a T cell), the antibody mediates antibody-dependent cell-mediated cytotoxicity (ADCC). Alternatively, the antibody may be part of an antibody-drug conjugate (ADC) whereby the antibody acts as a pathogenic immune cell-targeting component (e.g., T cell targeting) and the drug is a compound cytotoxic to the pathogenic immune cells (e.g., T cells).

In any aspect of the present invention, the at least one type of short chain fatty acid, ester or salt thereof is provided to the individual after the activity of, or number of, pathogenic immune cells (preferably T cells) has been reduced in the individual. Alternatively, the short chain fatty acid, ester or salt thereof is provided to the individual at the same time as reducing the number of pathogenic immune cells (preferably T cells) in the individual. Preferably, the short chain fatty acid, ester or salt thereof is provided to the individual for a longer duration than an agent is administered to reduce the activity of, or number of, pathogenic immune cells (preferably T cells) and restore immune tolerance.

In any aspect of the present invention, the at least one type of short chain fatty acid, ester or salt thereof is provided to the individual during and after the step of reducing the number of T cells in an individual.

Preferably, in any aspect of the invention, the at least one short chain fatty acid, ester or salt thereof, is a combination of at least two short chain fatty acids, esters, or salts thereof, such that the methods include providing in an individual, a therapeutically effective amount of a combination of two or more short chain fatty acids, esters or salts thereof is provided to the individual. In certain embodiments, the combination of two or more short chain fatty acids includes the combination of butyric acid and acetic acid.

In another aspect, the present invention provides a method for treating fatty liver disease in an individual, the method comprising, consisting essentially of or consisting of:
providing in the individual, a therapeutically effective amount of a combination of two or more short chain fatty acids, esters or salts thereof,

thereby treating the fatty liver disease in the individual.

In a further aspect, the present invention provides a method for preventing or delaying the onset of fatty liver disease in an individual, the method comprising, consisting essentially of or consisting of:
providing in the individual, a therapeutically effective amount of a combination of two or more short chain fatty acids, esters or salts thereof,

thereby preventing or delaying the onset the fatty liver disease in the individual.

The fatty liver disease may be associated, caused by or the result of alcoholism. Alternatively, the fatty liver disease may be associated, caused by or the result of a non-alcohol dietary cause (non-alcohol fatty liver disease, NAFLD). The NAFLD may be simple steatosis or may be simple steatosis including one or more signs of inflammation and fibrosis.

The fatty liver disease may be characterised by liver steatosis, elevated circulating or liver triglycerides and/or elevated circulating or liver cholesterol levels. Treating the fatty liver disease may be assessed by a reduction in the accumulation of fatty deposits in the liver, or a reduction in inflammation or fibrosis in a liver having fatty deposits.

In a further aspect, the present invention provides a method for treating non-alcoholic steatohepatitis (NASH) in an individual, the method comprising, consisting essentially of or consisting of:
providing in the individual, a therapeutically effective amount of a combination of two or more short chain fatty acids, esters or salts thereof,

thereby treating the NASH.

In another aspect, the present invention provides a method for preventing or delaying the onset of non-alcoholic steatohepatitis (NASH) in an individual, the method comprising, consisting essentially of or consisting of:
providing in the individual, a therapeutically effective amount of a combination of two or more short chain fatty acids, esters or salts thereof,

thereby preventing or delaying the onset of NASH.

In a further aspect, the invention provides a method for treating fatty liver disease in an individual, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,

thereby treating the fatty liver disease.

In a further aspect, the present invention relates to a method of preventing, delaying or reversing the onset or pathogenesis of NASH in an individual, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,

thereby preventing, delaying or reversing the onset or pathogenesis of NASH.

In any aspect of the invention, the individual may be determined to be at risk of developing NASH. For example, the individual may have inflammatory markers or other metabolic markers associated with a risk of developing NASH.

In a further aspect, the invention also provides a method of delaying the progression of, or treating a NASH in an individual, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,

thereby delaying the progression or treating the NASH.

In a further aspect, the invention also provides a method of reducing or treating inflammation in an individual at risk of, or having a NASH, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,

thereby reducing or treating inflammation in the individual.

Preferably, in the methods for treating or preventing fatty liver disease, including NAFLD and NASH, the pathogenic immune cells (preferably T cells) that are reduced in number, or have a reduced activity are positive for CXCR3 (e.g. Th1 T cells, or T cells). Preferably the reduction in activity or number of the pathogenic immune cells (preferably T cells) is by the administration of a CXCR3-depleting antibody.

In a further aspect, the invention provides a method for treating an autoimmune or inflammatory disease in an individual, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,

thereby treating the autoimmune or inflammatory disease.

Further, the present invention relates to a method of preventing or delaying the onset of an autoimmune or inflammatory disease in an individual, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,
thereby preventing or delaying the onset of the autoimmune or inflammatory disease.

In any aspect of the invention, the individual may be determined to be at risk of developing an autoimmune or inflammatory disease. For example, the individual may have autoantibodies or inflammatory markers associated with a risk of developing an autoimmune or inflammatory disease.

In a further aspect, the invention also provides a method of delaying the progression of, or treating an autoimmune or inflammatory disease in an individual, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,

thereby treating or delaying the progression or treating the autoimmune or inflammatory disease.

In a further aspect, the invention also provides a method of reducing or treating inflammation in an individual at risk of, or having an autoimmune disease or immune-mediated disorder, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,

thereby reducing or treating inflammation in the individual.

Reducing or treating inflammation may include reducing the proportion of one or more pro-inflammatory cytokines in the individual. Further, reducing or treating inflammation may include increasing the proportion of one or more anti-inflammatory cytokines in the individual.

In any aspect of the invention, reference to reducing the activity of or number of T cells in an individual includes modulating the balance of regulatory T cell function relative to effector T cell function. This includes modulating regulatory T cell function relative to effector T cell function.

Further, any reference herein to reducing the activity of, or number of, T cells in an individual, may also be a reference to modulating the balance of effector T cell function relative to regulatory T cell function or modulating effector T cell function relative to regulatory T cell function.

In a further aspect, the present invention also provides a method of preventing, reducing or treating autoimmunity in an individual at risk of, or having an autoimmune disease, the method comprising, consisting essentially of or consisting of:
reducing the activity of, or number of, pathogenic immune cells (preferably T cells) in an individual; and
providing in the individual, a therapeutically effective amount of at least one type of short chain fatty acid, ester or salt thereof,

thereby preventing, reducing or treating autoimmunity in the individual.

In any aspect relating to preventing, reducing or treating an autoimmune, inflammatory or autoinflammatory disease or condition, the pathogenic immune cell may be any one described herein, preferably an immune cell expressing CXCR3, for example a T cell or an NKT cell.

In any aspect or embodiment of the invention, “associated with” in the context of a disease or condition, may also be a reference to “caused by” or “the result of”.

Preventing, reducing or treating autoimmunity may include reducing the abundance or presence of one or more autoantibodies in the individual. The autoantibodies may be associated with a risk of autoimmune disease.

In any aspect of the present invention, the autoantibodies are associated with a risk of type 1 diabetes, including but not limited to islet autoantibodies, insulin autoantibodies and autoantibodies to glutamate decarboxylase (GAD) and islet-specific glucose-6-phosphatase catalytic subunit-related protein (IGPR).

In any aspect of the present invention, the disease requiring treatment is an autoimmune disease, is inflammation in an individual at risk of an autoimmune disease or is an inflammatory disorder or condition. Preferably the disease is selected from the group consisting of: type 1 diabetes, psoriasis, rheumatoid arthritis, inflammatory bowel disease (including ulcerative colitis and Crohn's disease), coeliac disease, autoimmune hepatitis, myocarditis, lupus nephritis, systemic lupus erythematosus (SLE), multiple sclerosis, graft versus host disease or primary biliary cirrhosis.

Preferably the disease requiring treatment is type 1 diabetes.

In any aspect of the present invention, the at least one type of short chain fatty acid, ester or salt thereof, includes a combination of two or more short chain fatty acids, esters or salts thereof, such as a combination of butyric acid and acetic acid, esters or salts thereof.

In a further aspect, the present invention provides a method of treating or delaying the progression of type I diabetes in an individual, the method comprising, consisting essentially of or consisting of:
reducing the activity of, or number of, T cells in an individual; and
providing in the individual, a therapeutically effective amount of butyric acid and/or acetic acid, esters or salts thereof,

thereby treating or delaying the progression of type I diabetes.

In a further aspect, the present invention also provides a method of preventing or delaying the onset of type I diabetes in an individual, the method comprising, consisting essentially of or consisting of:
reducing the activity of, or number of, T cells in an individual; and
providing in the individual, a therapeutically effective amount of butyric acid and/or acetic acid, esters or salts thereof,

thereby preventing or delaying the onset of type I diabetes.

In a further aspect, the present invention also provides a method of treating or delaying the progression of type I diabetes in an individual, the method comprising, consisting essentially of or consisting of:
reducing the activity of, or number of, T cells in an individual; and
providing in the large intestine of the individual, a therapeutically effective amount of butyric acid and/or acetic acid, esters or salts thereof,

thereby treating or preventing the type I diabetes.

In a further aspect, the present invention also provides a method of preventing or delaying the onset of type I diabetes in an individual, the method comprising, consisting essentially of or consisting of:
reducing the activity of, or number of, T cells in an individual; and
providing in the large intestine of the individual, a therapeutically effective amount of butyric acid and/or acetic acid, esters or salts thereof,

thereby preventing or delaying the onset of type I diabetes.

In a further aspect, the present invention also provides a method of reversing a symptom of type I diabetes and/or providing protection from relapse of type I diabetes in an individual, the method comprising, consisting essentially of or consisting of:
reducing the activity of, or number of, T cells in an individual; and
providing in the large intestine of the individual, a therapeutically effective amount of butyric acid and/or acetic acid, esters or salts thereof,
thereby reversing a symptom of type I diabetes and/or providing protection from relapse of type I diabetes.

Preferably, the disease requiring treatment is psoriasis. Accordingly, in a further aspect, the present invention provides a method of treating or delaying the progression of psoriasis in an individual, the method comprising, consisting essentially of or consisting of:
reducing the activity of, or number of, T cells, optionally pathogenic T cells, in an individual; and
providing in the individual, a therapeutically effective amount of butyric acid and/or acetic acid, esters or salts thereof,

thereby treating or delaying the progression of psoriasis.

In a further aspect, the present invention also provides a method of treating or delaying the progression of psoriasis in an individual, the method comprising, consisting essentially of or consisting of:
reducing the activity of, or number of, T cells, optionally pathogenic T cells in an individual; and
providing in the large intestine of the individual, a therapeutically effective amount of butyric acid and/or acetic acid, esters or salts thereof,

thereby treating or preventing the progression of psoriasis.

Preferably, the disease is multiple sclerosis. Accordingly, in a further aspect the present invention further provides a method of treating or delaying the progression of multiple sclerosis in an individual, the method comprising, consisting essentially of or consisting of:
reducing the activity of, or number of, T cells, optionally pathogenic T cells, in an individual; and
providing in the individual, a therapeutically effective amount of butyric acid and/or acetic acid, esters or salts thereof,

thereby treating or delaying the progression of multiple sclerosis.

In a further aspect, the present invention also provides a method of preventing or delaying the onset of multiple sclerosis in an individual, the method comprising, consisting essentially of or consisting of:
reducing the activity of, or number of, T cells, optionally pathogenic T cells in an individual; and
providing in the individual, a therapeutically effective amount of butyric acid and/or acetic acid, esters or salts thereof,

thereby preventing or delaying the onset of multiple sclerosis.

In a further aspect, the present invention also provides a method of treating or delaying the progression of multiple sclerosis in an individual, the method comprising, consisting essentially of or consisting of:
reducing the activity of, or number of, T cells, optionally pathogenic T cells, in an individual; and
providing in the large intestine of the individual, a therapeutically effective amount of butyric acid and/or acetic acid, esters or salts thereof,

thereby treating or preventing the multiple sclerosis.

In a further aspect, the present invention also provides a method of preventing or delaying the onset of multiple sclerosis in an individual, the method comprising, consisting essentially of or consisting of:
reducing the activity of, or number of, T cells, optionally pathogenic T cells in an individual; and
providing in the large intestine of the individual, a therapeutically effective amount of butyric acid and/or acetic acid, esters or salts thereof,

thereby preventing or delaying the onset of multiple sclerosis.

Preferably, in the methods for treating or preventing multiple sclerosis or psoriasis, the T cells that are reduced in number, or have a reduced activity are Th17 or Th22 T cells, or T cells positive for CCR6. Preferably, the reduction in activity or number of the T cells is by the administration of a CCR6-depleting antibody.

Preferably, the disease is GVHD. Accordingly, in a further aspect the present invention provides a method of treating or delaying the progression of GVHD in an individual, the method comprising, consisting essentially of or consisting of:
reducing the activity of, or number of, T cells, optionally pathogenic T cells in an individual; and
providing in the individual, a therapeutically effective amount of butyric acid and/or acetic acid, esters or salts thereof,

thereby treating or delaying the progression of GVHD.

In a further aspect, the present invention also provides a method of preventing or delaying the onset of GVHD in an individual, the method comprising, consisting essentially of or consisting of:
reducing the activity of, or number of, T cells, optionally pathogenic T cells in an individual; and
providing in the individual, a therapeutically effective amount of butyric acid and/or acetic acid, esters or salts thereof,

thereby preventing or delaying the onset of GVHD.

In a further aspect, the present invention also provides a method of treating or delaying the progression of GVHD in an individual, the method comprising, consisting essentially of or consisting of:
reducing the activity of, or number of, T cells, optionally pathogenic T cells in an individual; and
providing in the large intestine of the individual, a therapeutically effective amount of butyric acid and/or acetic acid, esters or salts thereof,

thereby treating or preventing the GVHD.

In a further aspect, the present invention also provides a method of preventing or delaying the onset of GVHD in an individual, the method comprising, consisting essentially of or consisting of:
reducing the activity of, or number of, T cells, optionally pathogenic T cells in an individual; and
providing in the large intestine of the individual, a therapeutically effective amount of butyric acid and/or acetic acid, esters or salts thereof,

thereby preventing or delaying the onset of GVHD.

Preferably, in the methods for treating or preventing GVHD, the T cells that are reduced in number, or have a reduced activity are Th1 T cells, activated T cells, or T cells positive for CXCR3. Preferably, the reduction in activity or number of the T cells is by the administration of a CXCR3-depleting antibody.

In any aspect of the present invention, the short chain fatty acid is selected from the group consisting of butyric acid, acetic acid and propionic acid.

Preferably, the at least one short chain fatty acid, ester or salt thereof, is a combination of at least two short chain fatty acids, esters, or salts thereof. The combination may be butyric acid and propionic acid, butyric acid and acetic acid, acetic acid and propionic acid, or acetic acid, butyric acid and propionic acid.

In any aspect of the invention, the combination of short chain fatty acids selected from acetic acid, butyric acid and propionic acid may further include additional short chain fatty acids selected from isobutyric acid, t-butyl carboxylic acid, pentanoic acid, hexanoic acid and the like. Further, the additional short chain fatty acid may be substituted with one to three substituents, such as a halogen (fluoro, chloro, bromo, iodo), cyano, hydroxyl, methoxy, keto and the like. Examples of useful substituted short chain fatty acids include hydroxyacetic acid, ketopropionic acid and 4,4-trifluorobutyric acid.

As used herein, the terms acetate, butyrate, propionate and the like, refer to the salt form of the free acid, or, depending on the physiological environment, the free acid itself. In context, it may also refer to an ester of the free acid.

The short chain fatty acids may be provided in the large intestine of the individual. In any embodiment of the present invention, the short chain fatty acids is provided in the colon of the individual. In any embodiment, the short chain fatty acids is provided systemically in the individual (i.e., in the peripheral blood circulation).

In any aspect of the present invention, the short chain fatty acids are provided in the individual by oral administration to the individual of a dietary agent or pharmaceutical composition including said short chain fatty acids. The dietary agent may include a carrier molecule covalently bonded to at least one short chain fatty acid, wherein the covalent bond is resistant to degradation in the small intestine of the individual but is hydrolysable in the colon to provide free fatty acid in the colon of the individual. Preferably, the carrier is a starch. Alternatively, the carrier may be a gum, oligosaccharide or pectin.

In any aspect of the present invention, the administration of the short chain fatty acids results in an increase in circulating levels of short chain fatty acids in the blood of the individual. In any embodiment of the invention, the increased circulating short chain fatty acid levels in the blood are sustained (i.e., not transient).

In any aspect of the present invention, the administration of the short chain fatty acids results in greater than, or equal to a 0.5-fold, 1-fold, 2-fold, 3-fold or 4-fold increase in the circulating levels of short chain fatty acids in the individual.

In further aspects of the invention, there is provided a kit for the treatment or prevention of an autoimmune disease or inflammatory disease, wherein the kit comprises, consists essentially of or consists of:
(a) an agent capable of reducing the activity or viability of a pathogenic immune cell (preferably a T cell); and
(b) at least one of species of short chain fatty acid selected from butyric acid, acetic acid, and propionic acid, including esters or salts thereof.

Preferably, the at least one species of short chain fatty acid includes a combination of least butyric acid and acetic acid.

Preferably, the short chain fatty acid(s) is provided in a pharmaceutical composition along with a pharmaceutically acceptable excipient, diluent or carrier.

The composition may be adapted for release of the short chain fatty acids into the large intestine of the individual.

The pharmaceutical composition may be adapted for release of the short chain fatty acids into the colon of the individual.

The pharmaceutical composition may be in the form of an oral dosage form including an enteric coating which is resistant to degradation in the stomach and small intestine. The enteric coating is preferably a digestion-resistant layer on the oral dosage form designed to release the short chain fatty acids into the lumen of the large intestine, preferably the colon.

The pharmaceutical composition may in the form of an oral dosage form, a suppository, or an injectable dosage form.

Preferably, the kit further comprises instructions for the administration of the agent and the pharmaceutical composition comprising at least one species of short chain fatty acid.

The present invention also includes the use of:

(a) an agent capable of reducing the activity or viability of a pathogenic immune cell (preferably a T cell), and

(b) at least one species of short chain fatty acid selected from butyric acid, acetic acid and propionic acid,

in the manufacture of a medicament for the treatment of or preventing or delaying the onset of an autoimmune disease or inflammatory disease.

In still further aspects, the invention provides a combination diet for use in the treatment or prevention of fatty liver disease, optionally wherein the disease is NASH, or for delaying the progression or onset of fatty liver disease, optionally wherein the disease is NASH, wherein the diet includes a combination of a first and a second dietary agent, the first agent including a carrier molecule covalently bonded to a butyric acid moiety, the second agent including a carrier molecule being covalently bonded to an acetic acid moiety, wherein in each agent, the moieties are bonded to the carriers by a bond that is hydrolysable in the colon of an individual, to give free butyric acid and free acetic acid.

In a further aspect, the present invention also provides for the use of two or more of butyric acid, acetic acid and propionic acid in the manufacture of a medicament for the treatment or prevention of fatty liver disease, preferably NASH.

In a further aspect, the present invention also provides a method for treating or delaying the progression of an autoimmune disease in an individual, wherein the autoimmune disease is preferably type 1 diabetes, the method comprising:
administering to the individual an agent that reduces the activity of, or number of, T cells in the individual;
administering to the individual a dosage form containing a therapeutically effective amount of a colonic composition, said composition consisting of:
   a core consisting of at least one species of short chain fatty acid, wherein the short chain fatty acid is selected from acetic acid, butyric acid and propionic acid, or a pharmaceutically acceptable salt or ester thereof; and
   at least one digestion-resistant layer covering said core,
   said digestion-resistant layer disintegrating in the colon; and
releasing the core within the lumen of the colon.

In a further aspect, the present invention also provides a method for preventing or delaying the onset of an autoimmune disease in an individual, wherein the autoimmune disease is preferably type 1 diabetes, the method comprising:
administering to the individual an agent that reduces the activity of, or number of, pathogenic immune cells (preferably T cells) in the individual;
administering to the individual a dosage form containing a therapeutically effective amount of a colonic composition, said composition consisting of:
   a core consisting of at least one species of short chain fatty acid, wherein the short chain fatty acid is selected from acetic acid, butyric acid and propionic acid, or a pharmaceutically acceptable salt or ester thereof; and
   at least one digestion-resistant layer covering said core,
   said digestion-resistant layer disintegrating in the colon; and
releasing the core within the lumen of the colon.

In a further aspect, the present invention also provides a combination composition, comprising, consisting essentially of, or consisting of:
an agent that is capable of reducing the activity of, or number of, pathogenic immune cells (preferably T cells) in an individual; and
a colonic composition comprising
   a core consisting of least one species of short chain fatty acid selected from butyric acid, acetic acid and propionic acid, or a pharmaceutically acceptable salt or ester thereof, and
   at least one digestion-resistant layer covering said core, said digestion resistant layer disintegrating in the colon.

The core may comprise or consist of at least two species of short chain fatty acids, preferably wherein the at least two species are butyric acid and acetic acid.

The dosage form may be in the form of a table or a capsule. Preferably, the core comprises both butyric acid and acetic acid, or a pharmaceutically acceptable salt or ester thereof. The dosage form is for use in the treatment of, or for delaying or preventing the onset of an autoimmune disease selected from type 1 diabetes, psoriasis, rheumatoid arthritis, inflammatory bowel disease, coeliac disease, autoimmune hepatitis, myocarditis, lupus nephritis, systemic lupus erythematosus (SLE), multiple sclerosis, and primary biliary cirrhosis. Preferably the autoimmune disease is type 1 diabetes. Preferably the autoimmune disease is type 1 diabetes.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 Diabetic NOD8.3 mice injected with anti-CD3 antibody and treated with medicinal supplement for 4 weeks. Individual blood glucose levels of pre-diabetic and newly diagnosed diabetic NOD8.3 mice treated with anti-CD3 monotherapy for one week, followed by SOFA medicinal supplement (15% HAMSA/15% HAMSB diet) for weeks two to four. Open symbols, hyperglycemic mice; filled symbols, normoglycemic mice. Mice receiving the combination therapy are protected from developing diabetes over the 4 week period.

FIG. 2 Schematic of treatment protocol to assess effect of combination of CXCR3+ cell depletion and SOFA therapy.

FIG. 3 H&E staining of liver sections, from mice receiving either: 1) control diet, 2) MCD diet plus isotype antibody, 3) MCD diet plus anti-CXCR3 antibody, 4) MCD diet plus HAMSA/HAMSB diet or 5) MCD diet plus anti-CXCR3 antibody followed by HAMSA/HAMSB diet. The sections show that mice in group 2 develop evidence of fatty liver disease and other characteristics of NASH compared to control (group 1). Mice receiving the MCD diet plus anti-CXCR3 antibody are partially protected from developing liver fibrosis. Mice receiving the MCD diet plus the HAMSA/HAMSB diet are also significantly protected from developing liver fibrosis. The combination of treatment with an anti-CXCR3 antibody and HAMSA/HAMSB diet provides greatest protection from fibrosis in mice fed an MCD diet.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

SCFAs produced from bacterial fermentation of fiber in the large intestine may promote gut health in numerous ways. For example, these acids are thought to be important for maintaining visceral function by increasing blood flow, and contribute to improved electrolyte and fluid absorption in diarrhoea, maintenance of low colonic pH to limit the growth of intestinal pathogens and also the modulation of colonic muscular activity. However, while diets high in acetylated or butyrylated high amylose maize starches (known as HAMSA and HAMSB, respectively) have been trialled in human populations with GI tract disorders, most efforts described previously for the correction of dysbiosis have relied on the use of probiotics.

Without being bound by any theory or mode of action it is believed that a reduction in pathogenic immune cells (such as T cells) while at the same time, or followed by, administering SCFAs results in a resetting of the immune system by both lowering pathogenic immune cell populations (such as pathogenic T cells, pathogenic B cells, pathogenic NK cell or pathogenic dendritic cells) and increasing regulatory T cells. Moreover, the present inventors have previously found that acetic acid and butyric acid act in different, yet complementary pathways to improve gut homeostasis, gut bacterial ecology and Treg numbers and function. For example, without wishing to be bound by theory, the inventors believe that butyric acid acts through a Treg associated pathway that is distinct from that described for acetate and which includes enhanced TGFβ production. Further, acetic acid is believed to be particularly useful in modifying the effects of antigen presenting cells, particularly B cells, thereby modifying the frequency of autoimmune T effector cells.

The present inventors have surprisingly found that a combination of pre-treatment aimed at reducing or depleting pathogenic immune cells (such as effector T cells), followed by a diet enriched for one or more SCFAs also plays an important role in the treatment and/or protection against autoimmune or autoinflammatory disease. More specifically, the inventors have found that reducing specific T-cell subsets including pathogenic T cells and providing a combination of one or more different SCFAs protects against the development of autoimmune and inflammatory disease thereby representing a novel treatment modality.

The short chain fatty acids are postulated to restore homeostatic systems important for normal immune responses, or tolerance induction. For instance, acetate or butyrate may enhance Treg activity and restore immunological tolerance. Thus the combination of both autoreactive T cell deletion, and enhancement of tolerance using a short chain fatty acids provides a unique approach to restoring immunological tolerance and reversal of autoimmune and inflammatory diseases.

In classical immune responses, pathogenic immune cells responses (such as effector T cell (Teff) responses) dominate over responses of T regulatory cells (Treg) resulting in antigen removal. Tolerance initiates with the same steps as the classical activation pathway (i.e., antigen presentation and T cell activation), but factors including, but not limited to, the abundance of antigen, the means by which it is presented to the T cell, and the relative availability of CD4+ cell help lead to the proliferation of a distinct class of lymphocytes called regulatory T cells. Just as effector T cells mediate classical immune responses, regulatory T cells mediate tolerogenic responses. However, unwanted or misdirected immune responses, such as those associated with allergy, autoimmune diseases, organ rejection, chronic administration of therapeutic proteins and the like, can lead to conditions in the body which are undesirable and which, in some instances, can prove fatal. The dominance or shifting of balance of regulatory T cells over pathogenic immune cells (such as effector T cells) results in antigen preservation and immunological tolerance.

Accordingly, in one aspect the present invention relates to a method of treating or preventing an autoimmune disease in an individual, the method including providing in the individual, a therapeutically effective amount of a combination of two or more short chain fatty acids, esters or salts thereof, thereby treating or preventing the autoimmune disease.

Short Chain Fatty Acids (SCFAs)

The short chain fatty acids (SCFAs) used in accordance with the present invention are selected from the group consisting of butyric acid, acetic acid and propionic acid. In any embodiment, either butyric acid or acetic acid is used in accordance with the present invention. Preferably the SOFA is butyric acid. In one embodiment, where a combination of SCFAs is used, the combination is of acetic acid and propionic acid. In another embodiment, the combination is propionic acid and butyric acid. In a particularly preferred embodiment, the SCFAs are butyric acid and acetic acid. In yet a further embodiment, all three species of SOFA are utilised.

The short chain fatty acids may be provided as sodium, potassium, calcium or magnesium salts. Where one of the short chain fatty acids is butyric acid, preferably, the salt is sodium butyrate. Where one of short chain fatty acids is acetic acid, preferably the salt is sodium acetate.

In some embodiments, the short chain fatty acid can be present as an ester of the carboxylic acid, with a branched or unbranched alkyl alcohol of one to 6 carbons. For example, the short chain fatty acid can be present as an ethyl ester, propyl ester, butyl ester, isopropyl ester, t-butyl ester, pentyl ester or hexyl ester.

The short chain fatty acids, or combination thereof may further comprise additional short chain fatty acids selected from isobutyrate, t-butyl carboxylate, pentanoate, hexanoate and the like. Further, the additional short chain fatty acid may be substituted with one to three substituents, such as a halogen (fluoro, chloro, bromo, iodo), cyano, hydroxyl, methoxy, keto and the like. Examples of useful substituted short chain fatty acids include hydroxyacetate, ketoporionate and 4,4-trifluorobutyrate.

When one of the short chain fatty acids is butyric acid, butyric acid is provided as a prodrug in the form of tributyrin, which is an ester comprised of butyrate and glycerol.

The SCFAs may be provided in an individual requiring treatment by any number of means known to the skilled person. For example, in one embodiment, the SCFAs are provided in a pharmaceutical formulation for oral, local or systemic administration, as further described herein. In a preferred embodiment, and as further described herein, the pharmaceutical formulation is adapted for delivery of the SCFAs to the large intestine, more particularly, the colon of the individual.

Alternatively, the SCFAs may be provided to the individual as part of the individuals' diet, whereby the SCFAs are provided for contact with the cells of the digestive tract upon digestion of a dietary agent in a desired region of the gastrointestinal tract. In a preferred embodiment, the dietary agent provides for release of the SCFAs in the colon, as further described herein.

Reducing the Activity of, or Number of, Pathogenic Immune Cells

The present invention involves reducing the number of pathogenic immune cells (e.g. T cells) in an individual. This may be by any means known to the skilled person or by any means described herein. For example, the step of reducing the number of pathogenic immune cells (preferably T cells) in an individual comprises, consists essentially of or consists of administering to the individual an agent capable of reducing the viability of a pathogenic immune cell, preferably a T cell. Administering such an agent to an individual results in a detectable reduction of the number of pathogenic immune cells, preferably T cells, more preferably peripheral T cells.

As used herein “pathogenic immune cells” are cells of the immune system that are associated with, or cause, any one of the diseases or conditions described herein. For example, the pathogenic immune cell may be any immune cell associated with, or that causes, an autoimmune, inflammatory or autoinflammatory disease, condition or response. The pathogenic immune cell may be any immune cell expressing CD3, CXCR3 and/or CCR6. The immune cells may be a T cell, a B cell, a natural killer (NK) cell (preferably an NKT cell) or a dendritic cell. The T cell may be any T cell described herein. For example, the T cell may be a Th1, Th17 or Th22 cell. Preferably, the T cell is a T cell that expresses CD3, CXCR3 and/or CCR6. The B cell may be any B cell described herein, preferably an inflammatory B cell. Typically, the B cell expressed CXCR3, or any other marker described herein. The NK cell may be any described herein, preferably an NKT cell. Typically, the NK expressed CXCR3, or any other marker described herein. The dendritic cell may be any described herein.

Preferably, the peripheral T cells are enriched for pathogenic cells, such as Th17 cells, or Th1 cells.

The agent may reduce the viability of the pathogenic immune cell (e.g. T cell) either directly or indirectly (i.e. mediate a reduction). Preferably, the agent is capable of depleting a pathogenic immune cell including a T cell, or population of T cells, when administered to the individual.

In any aspect of the present invention, a T cell may be any T cell. Preferably, the T cell is a Th1 cell, a Th17 cell, a Th22 cell or a natural killer T (NKT) cell. Preferably, the T cell is a T cell that expresses CD3, CXCR3 and/or CCR6.

The agent may be any compound or molecule that reduces the viability of a pathogenic immune cell (preferably T cell). In preferred forms, the inhibitor may be a small molecule chemical compound or interfering RNA (e.g. siRNA). The inhibitor may also be an antibody or antigen binding fragment thereof.

In a preferred form, the agent is an antibody that binds to a pathogenic immune cell (preferably T cell) and either directly or indirectly results in a reduction in viability of the T cell. In one example, upon binding to the pathogenic immune cell (preferably T cell) the antibody mediates antibody-dependent cell-mediated cytotoxicity (ADCC).

An antibody that binds to a pathogenic immune cell (preferably T cell) may bind to one or more cell surface molecules that are present on the surface of a pathogenic immune cell (preferably T cell) Preferably, the antibody binds to one or more cell surface molecules that are present on a pathogenic T cell, such as an effector and/or memory T cell, a Th1 cell, a Th 17 cell or a Th22 cell. Non-limiting examples of cell surface markers include CD3, CXCR3 and CCR6.

The skilled person will be familiar with various antibodies that are commercially available and which can be used to target T cells, including effector and/or memory T cells, a Th1 cell, a Th17 cell or a Th22 cell. The antibody may be a blocking (i.e., neutralising) antibody, whereby the antibody inhibits or reduces the activity of the T cell by binding to a relevant T cell receptor (e.g., CD3, CXCR3 or CCR6). Alternatively, the antibody may be a depleting antibody, whereby the antibody binds to a T cell and stimulates ADCC, resulting in a reduction or depletion in the number of T cells.

Examples of suitable antibodies for use in accordance with the present invention include:

Anti-CD3 antibodies: Muromab (Orthoclone® OKT3), Otelixizumab (TRX4), Teplizumab (PRV-031 formerly also known as MGA031 and hOKT3γ1(Ala-Ala)), Visilizumab (Nuvion®), Foralumab (TZLS-401).

Anti-CXCR3 antibodies: various anti-CXCR3 antibodies are commercially available, including but not limited to: 106 (anti-human CXCR3) or clone 173 (anti-mouse CXCR3), both available from Biolegend, BD Biosciences, AbCam or other supplier. Alternatively, the antibody may be an antibody as described in any of WO2018/119299, WO2013/109974, WO1998/011218 and WO2014/186842.

Anti-CCR6 antibodies: various anti-CCR6 antibodies are commercially available, including but not limited to: clone G034E3 (anti-human CCR6), available from Biolegend or Fluidigm. Other Anti-CCR6 antibodies are described in Robert, R. Ang, C & Sun, G. et al (2017) JCI Insight 2(15): e94821.

As used herein, the term “effector T cell” includes T cells which function to eliminate antigen (e.g., by producing cytokines which modulate the activation of other cells or by cytotoxic activity). The term “effector T cell” includes T helper cells (e.g., Th1, Th2, Th22 cells) and cytotoxic T cells. Th1 cells mediate delayed type hypersensitivity responses and macrophage activation while Th2 cells combat large extracellular pathogens (i.e. nematodes) but are also critical in the allergic response. As used herein, the term “T helper type 1 response” (Th1 response) refers to a response that is characterized by the production of one or more cytokines selected from IFN-γ, IL-2, TNF, and lymphotoxin (LT) and other cytokines produced preferentially or exclusively by Th1 cells rather than by Th2 cells. As used herein, a “T helper type 2 response” (Th2 response) refers to a response by CD4⁺ T cells that is characterized by the production of one or more cytokines selected from IL-4, IL-5, IL-6 and IL-10, and that is associated with efficient B cell “help” provided by the Th2 cells (e.g., enhanced IgG1 and/or IgE production and eosinophil production).

As used herein, the term “regulatory T cell” includes T cells which produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at levels less than or equal to that produced by Th1 or Th2 cells, e.g., an order of magnitude less than in Th1 or Th2 cells. Regulatory T cells can be found in the CD4+CD25+ population of cells. Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody, plus anti-CD28 antibody).

As used herein the phrase, “modulating the balance of regulatory T cell function relative to effector T cell function” or “modulating regulatory T cell function relative to effector T cell function” includes preferentially altering at least one regulatory T cell function (in a population of cells including both T effector cells and T regulatory cells) such that there is a shift in the balance of T effector/T regulatory cell activity as compared to the balance prior to treatment.

As used herein the phrase, “modulating the balance of effector T cell function relative to regulatory T cell function” or “modulating effector T cell function relative to regulatory T cell function” includes preferentially altering at least one effector T cell function (in a population of cells including both T effector cells and T regulatory cells) is altered such that there is a shift in the balance of T effector/T regulatory cell activity as compared to the balance prior to treatment.

In certain embodiments, the humanized anti-T cell antibodies described for use in a method herein are neutralizing antibodies. In certain exemplary embodiments, the antibodies have neutralizing activity in addition to enhanced effector function. The combined effects of T cell neutralization and T cell depletion may be advantageous whenever it is desirable to reduce or eliminate T cell-mediated effects.

For example, a “CXCR3 neutralizing antibody” binds to CXCR3 and blocks the activity of the receptor, such as the typical physiological and genetic responses resulting from CXCR3 ligands binding to CXCR3. A “CCR6 neutralizing antibody” binds to CCR6 and blocks the activity of the receptor, such as the typical physiological and genetic responses resulting from CCR6 ligands binding to CCR6. For example, a “CD3 neutralizing antibody” binds to CD3 and blocks the activity of the receptor, such as the typical physiological and genetic responses resulting from CD3 ligands binding to CD3.

Neutralizing activity may be complete (100% neutralization) or partial, e.g., approximately 10, 20, 30, 40, 50, 60, 70, 80, 90, 95 (or any percentage there between) or more neutralizing and will depend on various factors known to the skilled artisan, such as antibody concentration, affinity, and epitope as well as the particular assay used to evaluate neutralizing activity. The neutralizing activity of a neutralizing antibody may be shown by assays to measure inhibition of, e.g., ligand binding, GTP binding, calcium mobilization, cell chemotaxis, and/or receptor internalization. Numerous assays for determining the activity of neutralizing antibodies, and particularly neutralizing antibody, are known to the skilled artisan and may be readily adapted to verify that a particular antibody is neutralizing.

For example, in some embodiments, the neutralizing activity of an antibody may be assessed by a chemotaxis assay. The Neutralization Dose-50 (ND50) is defined as the concentration of antibody required to yield one-half maximal inhibition of the cell surface receptor-mediated recombinant human ligand response in a responsive cell line, at a specific ligand concentration. For example, to measure the ability of a CXCR3 antibody to block rhCXCL11 induced chemotaxis of hCXCR3 transfected BaF/3 cells, rhCXCL11 at 7 ng/mL is added to the lower compartment of a 96-well chemotaxis chamber (NeuroProbe, Cabin John, Md.). The chemotaxis chamber is then assembled using a PVP-free polycarbonate filter (5 μm pore size). Serial dilutions of the antibody (e.g., from 0.001 to 10000 μg/mL) and 0.25×10⁶ cells/well are added to the top wells of the chamber. After incubation for 3 hours at 37° C. in a 5% CO₂-humidified incubator, the chamber is disassembled and the cells that migrate through to the lower chamber are transferred to a working plate and quantitated using, for example, Resazurin Fluorescence.

Colvin et al., Mol Cell Biol 26: 5838-49 (2006) describe additional assays that can be used, in certain embodiments, to determine the neutralizing activity of neutralizing antibodies.

In certain embodiments, the antibodies disclosed herein can prevent or reduce calcium flux into the T cells that are targeted by the antibody. For example, in relation to a CXCR3-neturalising antibody, approximately 5×10⁶ cells are suspended in 2 mL of RPMI medium with 1% bovine serum albumin (BSA). Fifteen micrograms of Fura-2 (Molecular Probes, Eugene, Oreg.) are added and the cells are incubated at 37° C. for 20 min. The cells are washed twice in PBS and resuspended in 2 mL of calcium flux buffer (145 mM NaCl, 4 mM KCl, 1 mM NaHP04, 1.8 mM CaCl₂), 25 mM HEPES, 0.8 mM MgCl₂, and 22 nM glucose). Fluorescence readings are measured at 37° C. in a DeltaRAM fluorimeter (Photon Technology International, Lawrenceville, N.J.). Before and after the addition of chemokines (e.g., CXCL9, CXCL10, or CXCL11), intracellular calcium concentrations are recorded as the excitation fluorescence intensity emitted at 510 nm in response to sequential excitation at 340 nm and 380 nm and presented as the relative ratio of fluorescence at 340 nm to that at 380 nm.

In certain embodiments, receptor neutralization can be evaluated by measuring a reduction in receptor internalization. In some embodiments, receptor internalization assays may be performed by incubating about 2.5×10⁵ cells, such as CXCR3/300-19 cells, in RPMI medium with 1% BSA with various concentrations of CXCL10, CXCL11, or CXCL9 for 30 min at 37° C. The cells may then be washed with ice-cold flow cytometry staining buffer and subsequently analyzed for surface expression of CXCR3 using a PE-conjugated CXCR3 antibody.

The skilled person will be familiar with performed similar experiments for assessing the function of CCR6-neutralising antibodies or CD3-neutralising antibodies.

As assessed by any of the above assays, a neutralizing anti-CCR6, anti-CXCR3 or anti-CD3 antibody may have, in certain embodiments, an ND50 of approximately 0.01, 0.02, 0.05, 0.1, 0.2, 0.5, 1, 2, 3, 4, 5, 6, 7, 8, 9, 10, 15, 20, 40, 50, or 100 μg/mL. In particular embodiments, the ND50 may be 0.5-12 μg/mL, and in more particular embodiments,

Inhibition of cell migration, recruitment, or accumulation by an antibody or antigen-binding fragment provided herein can be assessed by any method known to those skilled in the art. Such methods can include, for example, analysis of biopsies by immunohistochemistry, flow cytometry, RT-PCR, etc., to assess the number of cells, such as CXCR3+, CCR6+, CD3+ cells, in one or more population of cells or one or more locations within the body or within an organ. Cell migration, recruitment, or accumulation can be inhibited by at least or about 10%, 15%, 20%, 25%, 30%, 35%, 40%, 45%, 50%, 55%, 60%, 65%, 70%, 75%, 80%, 85%, 90%, 95%, or more compared to the migration, recruitment, or accumulation in the absence of an antibody or antigen-binding fragment provided herein.

As used herein, “depletion” with respect to a T cell (i.e., any T cell as defined herein) refers to the reduction in activity or removal of these cells from a population of cells. As used herein, “depletion” with respect to CXCR3+ cells (i.e., cells expressing CXCR3 on their cell surface) refers to the reduction in activity or removal of these cells from a population of cells. As used herein, “depletion” with respect to CCR6+ cells (i.e., cells expressing CCR6 on their cell surface) refers to the reduction in activity or removal of these cells from a population of cells.

Reference to depletion includes complete or partial depletion. Further, depletion may be permanent or temporary, and may be to varying extents in magnitude and/or location. Depletion may be the result of cell death, such as by apoptosis or necrosis. Preferably the cell death in the context of an antibody is mediated via ADCC. Alternatively, an antigen binding site per se as described herein may have little or no capacity to reduce the activity or viability of a CXCR3+ cell but it may be conjugated to or associated with a compound that has the capacity to reduce the activity or viability of a cell. Typically, an antigen binding site that has little or no capacity to reduce the activity or viability of a CXCR3+ cell is conjugated to or associated with a cytotoxic compound.

Depletion can be assessed by measuring the number of relevant T cells in a population using any method known in the art (e.g., flow cytometry, immunohistochemistry, etc.), before and after exposure to an antibody or antigen-binding fragment provided herein, or in the absence and presence of an antibody or antigen-binding fragment provided herein. Following exposure to an antibody or antigen-binding fragment provided herein, the relevant population of T cells (e.g., Th1, Th17, Th22, CCR6+, CXCR3+, CD3+ etc) can be depleted by at least or about 10%, 15%, 20%, 25%, 30%, 35%, 40%, 45%, 50%, 55%, 60%, 65%, 70%, 75%, 80%, 85%, 90%, 95%, or more.

The present invention encompasses the use of antigen binding sites and/or antibodies described herein comprising a constant region of an antibody. This includes antigen binding fragments of an antibody fused to an Fc. The foregoing antigen binding sites can also be referred to as antigen binding domains of antibodies.

Preferably, an antigen binding site as described herein is an antibody or antigen binding fragment thereof. Typically, the antigen binding site is an antibody, for example, a monoclonal antibody.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2 and IgG3.

In one example, the Fc region of the constant region has an enhanced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In any aspect of the present invention, the antigen binding site comprises an Fc region that is engineered to have enhanced capacity to induce antibody-dependent cell-mediated cytotoxicity (ADCC). Preferably, the enhanced capacity to induce ADCC is conferred by mutation, deletion or modification of amino acids in the Fc region which interact with an Fc receptor. Preferably, the amino acids that are mutated, deleted or modified are at position 239, 330, and/or 332 as per SEQ ID NO:1 (where alanine is position 118) or at an equivalent position to 239, 330 and/or 332. Preferably, the amino acids are mutated to S239D, A330L and 1332E. Typically, the Fc comprises, consists essentially of or consists of an amino acid sequence shown in SEQ ID NO: 3.

In any aspect of the present invention, the antigen binding site comprises an Fc region that is not engineered to have a reduced capacity to induce antibody-dependent cell mediated cytotoxicity (ADCC). Preferably, there the amino acids at position 234, 235, and/or 331 as per SEQ ID NO: 1 (where alanine is position 118) or at an equivalent position to 234, 235 and/or 331 are not F, E and/or S respectively. In other words, the amino acid at position 234 is not F, at position 235 is not E and/or at 331 is not S.

In any aspect of the present invention, the antigen binding site does not comprise an Fc region comprising, consisting essentially of or consisting of an amino acid sequence as shown in SEQ ID NO: 2.

TABLE 1
Summary of amino acid sequences

| Antibody | | SEQ ID | |
|---|---|---|---|
| ID | Region | NO: | Amino acid or nucleotide sequence |
| hIgG1 | Fc region | 1 | ASTKGPSVFPLAPSSKSTSGGTAALGCLVKDY |
| | | | FPEPVTVSWNSGALTSGVHTFPAVLQSSGLYS |
| | | | LSSVVTVPSSSLGTQTYICNVNHKPSNTKVDK |
| | | | KVEPKSCDKTHTCPPCPAPELLGGPSVFLFPPKP |
| | | | KDTLMISRTPEVTCVVVDVSHEDPEVKFNWYV |
| | | | DGVEVHNAKTKPREEQYNSTYRVVSVLTVLHQ |
| | | | DWLNGKEYKCKVSNKALPAPIEKTISKAKGQPR |
| | | | EPQVYTLPPSRDELTKNQVSLTCLVKGFYPSDI |
| | | | AVEWESNGQPENNYKTTPPVLDSDGSFFLYSKL |
| | | | TVDKSRWQQGNVFSCSVMHEALHNHYTQKSLS |
| | | | LSPGK (bold is CH1, italics hinge region, |
| | | | underlined is CH2 and no formatting is CH3)) |
| 3SFc | Fc region | 2 | ASTKGPSVFPLAPSSKSTSGGTAALGCLVKDY |
| | | | FPEPVTVSWNSGALTSGVHTFPAVLQSSGLYS |
| | | | LSSVVTVPSSSLGTQTYICNVNHKPSNTKVDK |
| | | | KVEPKSCDKTHTCPPCPAPEFEGGPSVFLFPPKP |
| | | | KDTLMISRTPEVTCVVVDVSHEDPEVKFNWYV |
| | | | DGVEVHNAKTKPREEQYNSTYRVVSVLTVLHQ |
| | | | DWLNGKEYKCKVSNKALPASIEKTISKAKGQPR |
| | | | EPQVYTLPPSRDELTKNQVSLTCLVKGFYPSDI |
| | | | AVEWESNGQPENNYKTTPPVLDSDGSFFLYSKL |
| | | | TVDKSRWQQGNVFSCSVMHEALHNHYTQKSLS |
| | | | LSPGK |
| 3MFc | Fc region | 3 | ASTKGPSVFPLAPSSKSTSGGTAALGCLVKDY |
| | | | FPEPVTVSWNSGALTSGVHTFPAVLQSSGLYS |
| | | | LSSVVTVPSSSLGTQTYICNVNHKPSNTKVDK |
| | | | KVEPKSCDKTHTCPPCPAPELLGGPDVFLFPPKP |
| | | | KDTLMISRTPEVTCVVVDVSHEDPEVKFNWYV |
| | | | DGVEVHNAKTKPREEQYNSTYRVVSVLTVLHQ |
| | | | DWLNGKEYKCKVSNKALPLPEEKTISKAKGQP |
| | | | REPQVYTLPPSRDELTKNQVSLTCLVKGFYPSDI |
| | | | AVEWESNGQPENNYKTTPPVLDSDGSFFLYSKL |
| | | | TVDKSRWQQGNVFSCSVMHEALHNHYTQKSLS |
| | | | LSPGK |

In any aspect of the present invention, the antigen binding site per se as described herein may have little or no capacity to reduce the activity or viability of a CXCR3+ cell but it may be conjugated to or associated with a compound that has the capacity to reduce the activity or viability of a cell. For example, the Fc region may have a reduced ability or capacity to induce effector function, e.g., compared to a native or wild-type human IgG1 or IgG3 Fc region, however in this circumstance, the antigen binding site is conjugated to or associated with a cytotoxic compound.

As used herein, the term “tolerance” includes refractivity to activating receptor-mediated stimulation. Such refractivity is generally antigen-specific and persists after exposure to the tolerizing antigen has ceased. For example, tolerance is characterized by lack of cytokine production, e.g., IL-2 IFNγ, TNF or IL-17. Tolerance can occur to self antigens or to foreign antigens.

As used herein, the term “T cell” (i.e., T lymphocyte) is intended to include all cells within the T cell lineage, including thymocytes, immature T cells, mature T cells and the like, from a mammal (e.g., human). Preferably, Tcells are mature T cells that express either CD4 or CD8, but not both, and a T cell receptor. The various T cell populations described herein can be defined based on their cytokine profiles and their function.

As used herein, the term “naïve T cells” includes T cells that have not been exposed to cognate antigen and so are not activated or memory cells. Naïve T cells are not cycling and human naïve T cells are CD45RA+. If naïve T cells recognize antigen and receive additional signals depending upon but not limited to the amount of antigen, route of administration and timing of administration, they may proliferate and differentiate into various subsets of T cells, e.g. effector T cells.

As used herein, the term “memory T cell” includes lymphocytes which, after exposure to antigen, become functionally quiescent and which are capable of surviving for long periods in the absence of antigen. Human memory T cells are CD45RA−.

Human CD8+ T-cell types and/or populations can be identified using the phenotypic cell-surface markers CD62L, CCR7, CD27, CD28 and CD45RA or CD45RO (Sallusto F et al. Nature 1999, 401:708-712). As used herein, CD8+ T-cell types and/or populations have the following characteristics or pattern of expression of cell surface markers: Naive T cells are characterized as CD45RA+, CD27+, CD28+, CD62L+ and CCR7+; CD45RO+ Central Memory T cells are CD45RA−, CD27+, CD28+, CD62L+ and CCR7+; CD45RO+ Effector Memory T cells are defined by the lack of expression of these five markers (CD45RA−, CD27−, CD28−, CD62L− and CCR7-); and terminally differentiated Effector Memory CD45RA+ T cells are characterized as CD45RO+, CCR7−, CD27−, CD28−, CD62L−. Terminally differentiated Effector Memory cells further up-regulate markers such as CD57, KLRG1, CX3CR1 and exhibit strong cytotoxic properties characterized by their ability to produce high levels of Granzyme A and B, Perforin and IFNγ. Therefore, various populations of T cells can be separated from other cells and/or from each other based on their expression or lack of expression of these markers.

Different CD8+ T cell types can also exhibit particular functions, including, for example: secretion of IFN-γ; secretion of IL-2; production of Granzyme B; expression of FasL and expression of CD 107. However, while the expression pattern of cell surface markers is considered diagnostic of each particular CD8+ T cell type and/or population as described herein, the functional attributes of each cell type and/or population may vary depending on the amount of stimulation the cell(s) has or have received.

Effector functions or properties of T cells can be determined by the effector molecules that they release in response to specific binding of their T-cell receptor with antigen:MHC complex on the target cell, or in the case of CAR T-cells interaction of the chimeric antigen receptor, e.g. scFv, with the antigen expressed on the target cell. Cytotoxic effector molecules that can be released by cytotoxic CD8+ T cells include perforin, granzymes A and B, granulysin and Fas ligand. Generally, upon degranulation, perforin inserts itself into the target cell's plasma membrane, forming a pore, granzymes are serine proteases which can trigger apoptosis (a form of cell death), granulysin induces apoptosis in target cells, and Fas ligand can also induce apoptosis. Typically, these cytotoxic effector molecules are stored in lytic granules in the cell prior to release. Other effector molecules that can be released by cytotoxic T cells include IFN-γ, TNF-8 and TNF-α. IFN-γ can inhibit viral replication and activate macrophages, while TNF-8 and TNF-α can participate in macrophage activation and in killing target cells.

An activated T cell is a cell that is no longer in GO phase, and begins to produce one or more cytotoxins, cytokines and/or other membrane-associated markers characteristic of the cell type (e.g., CD8+) as described herein and is capable of recognizing and binding any target cell that displays the particular peptide:MHC complex or antigen alone on its surface and releasing its effector molecules.

T cells are divided into several major subclasses, including cytotoxic T cells, which kill virus-infected cells, as well as two classes of regulatory cells, called helper T cells (Th cells) and suppressor T cells, which act to modulate the activity of other immune cells. During chronic infections, Th cells develop into at least three phenotypically and functionally distinct effector populations, Th1, Th2 and Th17 T cells. Th1 cells produce IFN-.gamma and IL-2, which are commonly associated with cell-mediated immune responses against various intracellular pathogens, whereas Th2 cells produce cytokines such as IL-4, IL-5, and IL-13, that are crucial to control extracellular helminthic infections. Th17 cells produce isoforms of IL-17, and Th17 cells play an important role in maintaining mucosal barriers and contributing to pathogen clearance at mucosal surfaces.

Th1 cells have been associated with organ-specific autoimmune diseases, delayed-type hypersensitivity, and transplant rejection. Further, cytokines such as IL-12 and IL-4 have dominant roles in determining the outcome of Th differentiation into Th1 and Th2 subsets, respectively. For example, in Th1 cells, following the binding of IL-12 to its cognate receptor, STAT4 is activated, thereby leading to the production of IFN-γ. Accordingly, STAT4-deficient mice are defective in Th1 differentiation and do not respond to intracellular pathogens such as Listeria monocytogenes.

Th1 cells express various chemokine receptors such as CXCR3, CCR1, and CCR5. Generally, CXCR3 is considered to be one of the best markers of Th1 cells, although CXCR3 is expressed also by NKT cells and some B cells, particularly those associated with inflammation.

C-X-C motif chemokine receptor 3 (CXCR3) is a chemokine receptor primarily expressed on antigen experienced (memory), effector and activated T cells and is involved in recruiting these T cell subsets to sites of tissue inflammation in response to its primary ligands: CXCL9 (MIG), CXCL10 (IP-10), and CXCL11 (I-TAC). CXCR3 and CXCL10 are expressed in human T1D patients. In these patients, CXCL10 is expressed in the remaining insulin-producing beta cells in the islets. CXCR3 is expressed on invading T cells surrounding the islets. Similar expression patterns have been reproduced in non-obese diabetic (NOD) mice, a mouse model of type 1 diabetes. CXCR3 is expressed by dermal CD3+ lymphocytes and plasmacytoid dendritic cells, and its chemokine ligands, CXCL10 and CXCL9, are up-regulated, in psoriatic lesions.

CXCR3 is also expressed on infiltrating T cells present in certain types of inflamed tissues, while CXCL9, CXCL10 and CXCL11 are often produced by local cells in inflammatory lesions.

Upregulation of CXCR3 has been implicated in a range of autoimmune disorders. Largely absent from naive T cells, CXCR3 expression is upregulated upon activation with antigen. CXCR3 recruits these cells, including T helper 1 (Th1) cells, to sites of tissue inflammation in response to its primary ligands. Beta cells in the islets of Langerhans express CXCL9 and CXCL10 and T cells that have infiltrated the pancreas express CXCR3. Currently, there are no approved non-insulin treatment options for T1D. Agents are under investigation for the potential treatment of T1D and psoriasis to change the course of disease. Nevertheless, T1D carries a significant chronic disease burden and remains a major public health concern worldwide. A need exists for additional agents to treat or reduce the progression of various autoimmune disorders, particularly T1D, psoriasis, multiple sclerosis, autoimmune liver diseases, IBD, and other CXCR3-related disorders, such as vitiligo, alopecia and Graft Versus Host Disease.

In addition to Th1 and Th2 cells, it is now accepted that naïve CD4⁺ T cells can differentiate into Th17 or Th22 cells that secrete IL-17 or IL-22, respectively. Th1 cells were long considered to be the major effectors in multiple autoimmune diseases, while Th2 cells have been known to be involved in atopy and asthma. More recently, Th17 cells have been implicated as culprits in a plethora of autoimmune and other inflammatory diseases in mice and humans. Many of the disease states previously associated with Th1 cells, e.g., experimental autoimmune encephalomyelitis (EAE, a model for multiple sclerosis), collagen-induced arthritis, and some forms of colitis, were shown to be caused by IL-23-dependent Th17 cells or other IL-17-producing lymphoid cell types. An imbalance between Th17 and Treg cell function may be central in some of these diseases.

IL-22 was originally described in mice and humans as a cytokine characteristic of fully differentiated Th17 cells. Recently, however, a distinct subset of human skin-homing memory T cells has been shown to produce IL-22, but neither IL-17 nor IFNγ. Differentiation of IL-22 producing T cells, now named Th22 cells, could be promoted by stimulation of naive T cells in the presence of IL-6 and TNF or by the presence of plasmacytoid dendritic cells, and appears to be independent of RORC. The human Th22 cell population coexpresses the chemokine receptor CCR6 and the skin-homing receptors CCR4 and CCR10, which led to hypotheses that these cells may be important in skin homeostasis and pathology.

Th17 cells, the T helper cells that produce IL-17 (also referred to as IL-17A) and other pro-inflammatory cytokines, have been shown to have key functions in a wide variety of autoimmune disease models in mice and are thought to be similarly involved in human disease. In healthy humans, IL-17-secreting cells are present in the CD45RO+ CCR6+ populations of T cells from peripheral blood and gut. Th17 cells or their products have been associated with the pathology of multiple inflammatory or autoimmune disorders in humans. Th17 cells are broadly implicated in the pathogenesis of many common autoimmune disorders, including multiple sclerosis, rheumatoid arthritis, psoriasis, and inflammatory bowel disease.

Th22 cells are involved in skin immunity and remodeling, but are also implicated in cutaneous inflammatory conditions such as psoriasis.

Thh17 or Thh22 cell levels/viability can be assessed by standard techniques known to the skilled person, including cell surface staining for CCR6 and IL23R, ELISA for IL-17, IL-17F, and IL-22; intracellular staining for IL17, IL17F, IL22, or RORC, and Western Blot for IL-17, IL-17F, IL23R, RORC, IL22.

Conditions for Prevention and/or Treatment

The methods of the present invention are useful for the prevention and/or treatment of any disease associated with, or caused by, pathogenic immune cells.

The methods of the present invention are useful for the prevention and/or treatment of any disease which results in an increased autoimmune and/or autoinflammatory response in one or more regions of the body.

The methods of the present invention are particularly useful for the treatment and/or prevention of inflammation associated with, caused by or resulting from autoimmune disease.

The methods of the present invention are therefore useful for treating diseases associated with, mediated by or caused by dysfunctional/ineffective regulatory T cell function, expanded autoreactive T effector cells, and/or B cell dysfunction. Diseases which may be prevented and/or treated in accordance with the present invention are autoimmune diseases, including, for example, an autoimmune disease selected from the group consisting of type 1 diabetes, psoriasis, rheumatoid arthritis, inflammatory bowel disease, coeliac disease, autoimmune hepatitis, myocarditis, lupus nephritis, systemic lupus erythematosus (SLE), primary biliary cirrhosis and multiple sclerosis.

As used herein, the phrase “autoimmune disease” refers generally to those diseases characterized by the failure of one or more B- and/or T-cell populations, or gene products thereof, to distinguish between self and non-self antigenic determinants.

As used herein, the phrase “autoinflammatory disease” or “autoinflammatory diseases” (AIDs) refers generally to those diseases characterized by the dysregulated secretion of pro-inflammatory cytokines and consequent damage to host tissues.

AIDs and systemic autoimmune diseases (ADs), share some characteristics: they start with the prefix “auto” to define a pathological process directed against self; they are systemic diseases, frequently involving musculoskeletal system; both include monogenic and polygenic diseases. From the pathogenetic point of view, they are characterized by a chronic activation of immune system, which eventually leads to tissue inflammation in genetically predisposed individuals. Nevertheless, the specific effectors of the damage are different in the two groups of diseases: in AIDs the innate immune system directly causes tissue inflammation, whereas in ADs the innate immune system activates the adaptive immune system which, in turn, is responsible for the inflammatory process.

As used herein, “preventing”, “prevention”, “preventative” or “prophylactic” refers to keeping from occurring, or to hinder, defend from, or protect from the occurrence of a condition, disease, disorder, or phenotype, including an abnormality or symptom. An individual in need of prevention may be prone to developing an autoimmune disease. For example, preventing an autoimmune disease in accordance with the present invention includes preventing or delaying the onset of said disease in an individual identified as being at risk of developing the disease. An individual may be identified as being at risk either by way of genetic testing, analysis of environmental factors, family history or other factors.

An individual in need of treatment may be one diagnosed with, or at risk of developing, any one of the autoimmune diseases described herein. The term ‘treatment’, or “treating” as used herein includes minimising the progression or delaying the progression of a disease. For example, the methods of the present invention may be useful in preventing the onset of disease in an individual showing early signs of disease. As an example, in the context of type I diabetes, an individual with early signs of the disease may show signs of pancreatic islet damage, or have islet autoantibodies that are markers for pancreatic damage, but does not yet have abnormal glucose tolerance. Further progression of the disease may include abnormal glucose tolerance, but not yet requiring insulin treatment. The skilled person will appreciate that the methods of the present invention are useful for the treatment of type I diabetes in any of these contexts.

Treatment of type I diabetes includes reversing a symptom of type I diabetes, providing protection from relapse of type I diabetes or reducing blood glucose levels.

In addition to primates, such as humans, a variety of other mammals can be treated according to the methods of the present invention. For instance, mammals including, but not limited to, cows, sheep, goats, horses, dogs, cats, guinea pigs, rats or other bovine, ovine, equine, canine, feline, rodent or murine species can be treated.

Type 1 Diabetes

The methods of the present invention have particular utility in the prevention and treatment of type 1 diabetes. The skilled person will be familiar with methods for identifying an individual requiring prevention or treatment for T1D, including an individual for whom there is a need to delay the onset or progression of the disease.

The development of T1D and progression of the disease is a multi-stage process. In 2015, the JDRF (Juvenile Diabetes Research Foundation), the Endocrine Society and the American Diabetes association, released a scientific statement, establishing the adoption of a staging classification for the development of T1D (Insel et al., 2015, Diabetes Care, 38:1964-1974, the entire contents of which are herein incorporated in their entirety). Briefly, the stages are:

Pre-stage 1: genetic susceptibility and genetic risk of T1D

Stage 1: autoimmunity/normoglycaemia/presymptomatic;

Stage 2: autoimmunity+/dysglycaemia/presymptomatic; and

Stage 3: autoimmunity+/dysglycaemia/symptomatic T1D.

The HLA region on chromosome 6 accounts for about 30-50% of the genetic risk of T1D, with the greatest association with HLA class II haplotypes DRB1*0301-DQB1*0201 (DR3-DQ2) and DRB1*0401-DQB1*0302 (DR4-DQ8). The genotype associated with the highest risk for T1D is the heterozygous DR3/4 genotype. HLA class II DRB1*1501 and DQA1*0102-DQB1*0602 confer disease resistance, at least in children younger than 12 years of age.

The remaining genetic risk for T1D can be attributed to the approximately 50 non-HLA genes or loci identified via candidate gene and genome-wide association study approaches, each with modest to small effects on disease risk. The highest non-HLA genetic contribution arises from the INS, PTPN22, CTLA4, and IL2RA genes, with the latter three genes also contributing to susceptibility to other autoimmune diseases. Non-HLA genetic contribution may be acting through immune regulation, although the recent demonstration of gene expression commonly in pancreatic islets and the alternative splicing of several of these gene products in cytokine-stimulated islets have raised the question of whether some of these genes may in part be acting in the β-cell.

Stage 1 represents individuals who have developed two or more T1D-associated islet autoantibodies but are normoglycemic. For children who were screened for genetic risk at birth and reach this stage, the 5-year and 10-year risks of symptomatic disease are approximately 44% and 70%, respectively, and the lifetime risk approaches 100%. The risk at this stage is quite similar in genetically at-risk children and in relatives of individuals with type 1 diabetes. Stage 1 is defined as the presence of two or more islet autoantibodies to insulin, GAD65, IA-2, and/or ZnT8. The mechanisms leading to β-cell autoimmune reactivity have not been completely elucidated. (Pro)insulin, GAD65, IA-2, and ZnT8 and their peptides have been identified as target antigens in T1D. Islet autoantibodies can be measured with standardized, sensitive, and high-throughput assays.

The number of detectable islet autoantibodies correlates with risk. The rate of progression to symptomatic disease in the presence of two or more islet autoantibodies is associated not only with the number of autoantibodies detected and the age of autoantibody seroconversion but also with the magnitude of the autoimmunity titer, affinity of the autoantibody, and the type of autoantibody. Higher titers of insulin and IA-2 autoantibodies are associated with earlier onset of symptomatic type 1 diabetes. The presence of IA-2 or ZnT8 autoantibodies is associated with faster progression to symptomatic disease compared with when both are absent. In first-degree relatives of individuals with type 1 diabetes, IA-2 and/or ZnT8 autoantibody seroconversion is associated with a 5-year progression rate to diabetes of 45%, and the presence of either of these autoantibodies is detected in 78% of progressors to symptomatic disease. Thus, the presence of two or more autoantibodies is used as the major criterion for stage 1. The majority of individuals (85%) with a single autoantibody do not progress to overt symptomatic type 1 diabetes within 10 years. However, some single autoantibody subjects can progress, and progression appears to occur more frequently in children aged <5 years, if the single autoantibody is directed to IA-2 or if the single autoantibody displays higher affinity.

Stage 2 is defined as the presence of β-cell autoimmunity with dysglycemia and is presymptomatic. Stage 2, like stage 1, includes individuals with islet autoantibodies but whose disease has now progressed to the development of glucose intolerance, or dysglycemia, that arises from loss of functional β-cell mass. Dysglycemia in this stage of T1D has been defined in several studies by impaired fasting plasma glucose of 00 mg/dL (≥5.6 mmol/L) or ≥110 mg/dL (≥6.2 mmol/L), impaired glucose tolerance with 2-h plasma glucose with a 75-g oral glucose tolerance test (OGTT) of ≥140 mg/dL (≥7.8 mmol/L), high glucose levels at intermediate time points on OGTT (30, 60, 90 min levels of 200 mg/dL [≥11.1 mmol/L]), and/or HbA_{1c}≥5.7% (≥9 mmol/mol).

Stage 3 represents manifestations of the typical clinical symptoms and signs of diabetes, which may include polyuria, polydipsia, weight loss, fatigue, diabetic ketoacidosis (DKA), and others.

In any embodiment of the invention, the individual may have residual endogenous insulin production although may be classified as falling within Stage 3 (i.e., having clinical symptoms of T1D in addition to dysglycaemia and autoimmunity). The present invention also contemplates methods for treating T1D in such individuals, including for example, to reduce or reverse symptoms of active disease, or enable the individual to be considered “in remission” with respect to symptomatic T1D.

Thus, in any embodiment of the present invention, an individual who is considered at risk of T1D, shows early signs of T1D or who will benefit from the treatments and therapies described herein includes an individual who is classified according to any of the stages 1-3 outlined above, or in “pre-stage 1”.

It will be well within the purview of the skilled person to be able to perform the relevant clinical tests to determine whether an individual is at risk of the development of T1D (e.g. using the classification system described herein), and is therefore an individual for whom delay or prevention of T1D may be achieved using the methods and compositions of the present invention.

For example, the skilled person will be familiar with methods for screening for the presence of autoantibodies in an individual, including autoantibodies to one or more β-cell specific antigens. Autoantibodies which may be screened for in accordance with the methods of the present invention, are those which are raised against antigens selected from the group consisting of: Carboxypeptidase H, Chromogranin A, Glutamate decarboxylase (GAD65), Imogen-38, Proinsulin/Insulin, Insulinoma antigen-2 and 2β, Islet-specific glucose-6-phosphatase catalytic subunit related protein (IGRP) zinc transporter 8 (ZnT8), and Proinsulin. Preferably, to determine whether an individual is at risk of T1D, the presence of antibodies raised to Islet Cells, Glutamic Acid Decarboxylase, Insulin Autoantibodies (including proinsulin autoantibodies), and IA-2A will be determined.

The skilled person will also be familiar with methods for determining whether an individual is “normoglycaemic” or has symptoms of “dysglycaemia”, which may assist in determining the stage of T1D development, and whether the individual is displaying symptoms of T1D. Methods for determining blood glucose levels, including after oral glucose challenge, will be familiar to the skilled person.

As used herein, “normoglycaemia” (or euglycaemia) refers to a normal blood glucose level (i.e., one which is not dysglycaemic or hyperglycaemic as herein defined).

As used herein, dysglycemia refers to impaired fasting plasma glucose of 100 mg/dL (≥5.6 mmol/L) or ≥110 mg/dL (≥6.2 mmol/L), impaired glucose tolerance with 2-h plasma glucose with a 75-g oral glucose tolerance test (OGTT) of ≥140 mg/dL (≥7.8 mmol/L), high glucose levels at intermediate time points on OGTT (30, 60, 90 min levels of ≥200 mg/dL [≥11.1 mmol/L]), and/or HbA_{1c}≥5.7% (39 mmol/mol).

The term “hyperglycaemia” as used herein refers generally to blood glucose levels that are above normal. Hyperglycaemia can be determined by any measure accepted and utilized by those of skill in the art. Currently, in humans, normal blood glucose is considered to be between about 70 and 120 mg/dl (3.9-6.6 mmol/L), but varies depending on the fasting state. Before a meal, blood glucose can range from about 80 to 120 mg/dl (4.4-6.6 mmol/L), whereas two hours after a meal, blood glucose can be at or below about 180 mg/dl (10 mmol/L). Additionally, in fasted individuals, normal blood glucose is below about 110 mg/dl (6.1 mmol/L). A subject having a blood glucose value of about 126 mg/dl (7 mmol/L) or greater is generally considered hyperglycaemic, and a subject whose blood glucose is above about 200 mg/dl (11.1 mmol/L) is generally considered diabetic.

The skilled person will also be able to determine whether an individual is “pre-symptomatic” or has commenced exhibiting symptoms of clinical manifestations of T1D. For example, symptoms of polyuria, polydipsia, weight loss, fatigue and diabetic ketoacidosis are symptoms of T1D. As used herein, polyuria refers to excessive or abnormally large production or passage of urine (greater than 2.5 or 3 L over 24 hours in adults). Frequent urination is sometimes included by definition but is nonetheless usually an accompanying symptom.

As used herein, polydipsia refers to excessive thirst, and may also be accompanied by dry mouth.

Diabetic ketoacidosis (DKA) is related to hyperglycaemia, is associated with illness or very high blood glucose levels in type 1 diabetes and can be a sign of insufficient insulin production. In the absence of sufficient insulin, the body burns fat for energy instead, which may lead to accumulation of ketones in the blood (and which also may appear in the urine). DKA generally refers to high blood glucose levels and moderate to heavy ketones in the urine. Other symptoms or indicators of DKA include rapid breathing, flushed cheeks, abdominal pain, sweet acetone (similar to paint thinner or nail polish remover) smell on the breath, vomiting and dehydration.

The skilled person will also be familiar with other methods for determining whether an individual is at risk of or is displaying early signs of T1D such as by measuring the frequency of CD4+ or CD8+ T-cells which are specific for a pancreatic antigen. This may include detecting the frequency of autoreactive T cells, including CD8+ T cells to one or more of proinsulin, islet-specific glucose-6-phosphatase catalytic subunit related protein (IGRP), glutamate decarboxylase (GAD), islet-antigen 2 (IA-2) and zinc transporter 8 (ZnT8).

Fatty Liver Disease and NASH

The present invention also relates to methods for preventing the onset of, preventing the progression of, or treating fatty liver disease in an individual who is at risk of developing fatty liver disease or is displaying symptoms of fatty liver disease.

In certain preferred embodiments, the present invention provides methods for preventing the onset of, preventing the progression of, or treating NASH in an individual who is at risk of developing NASH or is displaying symptoms of NASH.

The methods of the present invention may find utility in preventing or delaying the development of symptoms of NAFLD including elevated liver triglycerides and increase liver weight. Fatty liver disease that develops in the absence of alcohol abuse is recognized increasingly as a major health burden, in particular if an inflammatory component is involved such as in non-alcoholic steatohepatitis (NASH). Estimates based on imaging and biopsy studies suggest that about 20% to 30% of adults in the United States and other Western countries have excess fat accumulation in the liver. About 10% of these individuals, or fully 2% to 3% of adults, are estimated to meet current diagnostic criteria for NASH. Sustained liver injury leads to progressive fibrosis and cirrhosis in about 30% of NASH patients. The diagnostic criteria for NASH continue to evolve and rely on the histologic findings of steatosis, hepatocellular injury (ballooning, Mallory bodies), and the pattern of fibrosis.

As used herein, NAFLD refers to non-alcoholic fatty liver disease. NAFLD, which is considered to be a non-life threatening to disease, is to be distinguished from NASH which is a more severe form of NAFLD, and associated with increased mortality. The terms “non-alcoholic fatty liver disease” or “NAFLD”, “non-alcoholic steatohepatitis” or “NASH”, and “Simple Steatosis” are each used in the sense which are currently admitted by the scientific community. In general, NAFLD exists as a histological spectrum of changes. All of the stages of NAFLD have in common the accumulation of fat in the liver cells (steatosis). Simple steatosis refers to the hepatic steatosis in the absence of significant inflammation and hepatocellular damage, whereas NASH demonstrates inflammation and hepatocellular damage and sometimes fibrosis.

The skilled person will be familiar with methods for determining if an individual has, or is at risk of developing non-alcoholic steatohepatitis (NASH). In particular, the skilled person will be familiar with methods for determining if an individual has symptoms or signs of non-alcoholic fatty liver disease, including whether the individual has, or is at risk of any of the subtypes of fatty liver disease such as steatosis, NASH or NAFDL, and, in particular, for the differentiation of the life threatening NASH from the less severe NAFLD.

The term “fatty liver disease” is well known in the art. Preferably, the term refers to an impairment of the liver. Preferably, said impairment is the result of a surplus of triacylglyceride that accumulate in the liver and form large vacuoles. The symptoms accompanying fatty liver disease are well known from standard text books of medicine such as Stedman's or Pschyrembel. Fatty liver disease may result from alcohol abuse, diabetes mellitus, nutritional defects and wrong diets, toxicity of drugs or genetic predisposition. Fatty liver disease as used in accordance with the present invention also include the more severe forms thereof and, in particular, steatosis, NASH or NAFDL. Symptoms accompanying these diseases are also well known to the physicians and are described in detail in standard text books of medicine.

Liver biopsy has remained the criterion standard or “gold standard” in the evaluation of the etiology and extent of disease of the liver such as NAFLD and NASH. Percutaneous liver biopsy is the preferred method to determine NAFLD and to differentiate NASH from NAFLD. Other biopsy methods are typically even more invasive and include transvenous and laparoscopic liver biopsy. The American Gastroenterological Association has published detailed recommendations on how to grade NAFLD comprising NASH into macrovescicular steatosis grades, necroinflammatory activity grades and fibrosis stages (American Gastroenterological Association 2002, Gastroenterology 123: 1705-25; Brunt 1999, Am J Gastroenterol. 94: 2467-74, Brunt 2010, Nat Rev Gastroenterol Hepatol. 7:195-203).

While simple steatosis appears to be a relatively benign condition, it may progress to NASH over time. Accordingly, a person at risk of NASH may be a person diagnosed as having simple steatosis. In addition to its association with cardiovascular complications, NAFLD can lead to liver related morbidity and mortality. The risk of developing cirrhosis is higher in the presence of NASH, which is more likely in the presence of the following features:

type 2 diabetes mellitus (T2DM)

obesity (body mass index [BMI]>30 kg/m2)

age more than 50 years

serum aminotransferases (ALT or AST) more than two times the upper limit of normal.

Accordingly, a person may be considered to be at risk of NASH if they are diagnosed with NAFLD and have one or more of the above features. A definitive diagnosis of NAFLD depends on three factors:
1) evidence of fatty infiltration from either imaging (ultrasound, magnetic resonance imaging [MRI]) or histology (liver biopsy)
2) exclusion of significant alcohol consumption
3) exclusion of other causes of hepatic steatosis (eg. medications, surgery, metabolic disorders).

In addition to the above, signs of cirrhotic complications may also be considered, including signs of portal hypertension (splenomegaly, increased portal vein size, varices) or other complications such as HCC, portal vein thrombosis, or ascites. The risk of fibrosis and progressive liver disease in NAFLD increases with severity of insulin resistance. The number of metabolic syndrome features (obesity, combined hyperlipidemia, diabetes mellitus (type II), and high blood pressure) can be used to estimate risk of insulin resistance. The presence of three or more features of the syndrome, especially if these include central adiposity and type 2 diabetes mellitus (T2DM) are predictive of the presence of NASH rather than simple steatosis. In addition, family history plays a role: an individual with a first degree relative with T2DM has a 90% chance of developing T2DM, and therefore NASH. Central adiposity can be assessed using waist circumference measured at the narrowest point mid-way between the lowest rib and the iliac crest at the end of expiration with the patient standing.

Non-invasive tools for estimating the degree of fibrosis include transient elastography (FibroScan®), acoustic radiation force impulse (ARFI), and non-invasive biomarker algorithms such as NAFLD Fibrosis Score, FibroTest and Hepascore.

The skilled person will also be familiar with methods for determining whether an individual has been successfully treated so as to prevent the further progression of NASH, reversal of symptoms of NASH or prevention of the development of NASH in an at-risk individual. Successful treatment or prevention can be determined by measuring for an improvement, or reduction in the rate of progression or worsening of any one or more of the symptoms described above associated with NASH.

Psoriasis

The present application also relates to methods for preventing or treating psoriasis in an individual.

The skilled person will be familiar with various methods for diagnosing or identifying an individual having psoriasis. Moreover the skilled person will be familiar with methods for distinguishing between forms of psoriasis, for example by histological classification. Methods for diagnosing and classifying psoriasis and other skin disorders are known to the skilled person.

Where the skin disorder is psoriasis, the psoriasis may be pustular or non-pustular psoriasis.

Examples of non pustular psoriasis that can be treated in accordance with the present invention, include, psoriasis vulgaris, guttate psoriasis (also called eruptive psoriasis), inverse psoriasis (also called flexural psoriasis), or erythrodermic psoriasis (psoriatic erythroderma).

The skin disorder may be localised or generalised pustular psoriasis. Where the psoriasis is generalised psoriasis, the condition may be annular (circinate) pustular psoriasis which is a subacute form of generalised pustular psoriasis. Alternatively, the condition may be Vin Zumbusch pustular psoriasis. Other forms of pustular psoriasis include pustolosis palmaris et plantaris (palmaplantar pustulosis), acrodermatitis continua, subcorneal pustular dermatitis, and impetigo herptiformis.

Atopic dermatitis may be a form of eczema selected from the group consisting of endogenous eczema, flexural eczema, infantile eczema, and it may also be known as “prurigo Besnier,” “neurodermitis,” or “prurigo diathesique”.

The skilled person will be familiar with methods for determining the successful treatment of psoriasis following a treatment according to a method described herein. For example, successful treatment or determining the efficacy of a treatment described herein, may include using the Psoriasis Area Severity Index (PASO.

The Psoriasis Area and Severity Index (PASI) is used by dermatologists to assess psoriasis disease intensity. This index is based on the quantitative assessment of three typical signs of psoriatic lesions: erythema, infiltration, and desquamation, combined with the skin surface area involvement. Since its development in 1978, this instrument has been used throughout the world by clinical investigators (Fredriksson T, Petersson U: Severe psoriasis—oral therapy with a new retinoid. Dermatologica 1978; 157: 238-41.)

PASI is indicated as PASI 50 (a 50 percent improvement in PASI from baseline), PASI 75 (a 75 percent improvement in PASI from baseline), PASI 90 (a 90 percent improvement in PASI from baseline), and PASI 100 (a 100 percent improvement in PASI from baseline). The efficacy of a treatment of psoriatic arthritis in a patient population who has psoriasis, may be evaluated by determining the percentage of the patient population in whom a PASI 50, PASI 75, PASI 90, or PASI 100 response has been achieved following administration of the treatment.

The Physicians Global Assessment (PGA) is used to assess psoriasis activity and follow clinical response to treatment. It is a six-point score that summarizes the overall quality (erythema, scaling and thickness) and extent of plaques relative to the baseline assessment. A patient's response is rated as worse, poor (0-24%), fair (25-49%), good (50-74%), excellent (75-99%), or cleared (100%).

The DLQI is an additional validated instrument used to assess dermatologic-related functional limitations. Characteristics of the DLQI include:
ten items on an overall scoring range of 0-30; higher scores represent greater quality of life impairment and lower scores represent lower quality of life impairment;
well-established properties of reliability and validity for the DLQI total score in a dermatology setting (see Badia et al. (1999) Br J Dermatol 141:698; Finlay et al. (1994) Clin Exp Dermatol 19:210; and Shikier et al. (2003) Health and Quality of Life Outcomes 1:53);
six subcategories: symptoms and feelings; daily activities; leisure; work/school;
personal relationships; and treatment;
all data are observed values. Patients who discontinued before the time point were not included in this analysis.

Ranges of DLQI scores can be evaluated for their correspondence to categories of disease impact.

The PASI, PGA, and DLQI scores may be used as an index for measuring efficacy of a treatment as described herein, in a patient population having psoriasis, where attaining a certain percentage of patients within a population who were administered the treatment and who maintain clinical remission, i.e. PASI <50 or PASI <75, indicates that the treatment is effective for treating of psoriasis.

In one embodiment, the present invention provides a method for determining whether a treatment described herein is effective for treating psoriasis.

The efficacy of a treatment for treating psoriasis in a patient population, i.e., PASI 75 response (also referred to herein as a PASI/PASI 75 score), may be evaluated by determining the percentage of the patient population in treatment of psoriasis has been effective following administration of the treatment.

Graft Versus Host Disease

The methods of the present invention also have utility in treating or preventing the onset or progression of GVHD, or reducing the severity of GVHD in an individual requiring cell transplantation.

GVHD is an inflammatory disease initiated by T cells in the donor graft that recognize histocompatibility and other tissue antigens of the host and GVHD is mediated by a variety of effector cells and inflammatory cytokines. GVHD presents in both acute and chronic forms. The most common symptomatic organs are the skin, liver, and gastrointestinal tract, including the oral cavity and oropharyngeal regions. GVHD may involve other organs such as the lung.

Treatment of GVHD is generally only 50-75% successful; the remainder of patients generally do not survive. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of HLA-matched unrelated donor marrow, and in a higher percentage of recipient of HLA-mismatched marrow. Patients with mild intestinal GVHD present with anorexia, nausea, vomiting, abdominal pain and diarrhea, whereas patients with severe GVHD are disabled by these symptoms. If untreated, symptoms of intestinal GVHD persist and often progress; spontaneous remissions are unusual. In its most severe form, GVHD leads to necrosis and exfoliation of most of the epithelial cells of the intestinal mucosa, a frequently fatal condition. The symptoms of acute GVHD usually present within 100 days of transplantation. The symptoms of chronic GVHD usually present somewhat later, up to three years after allogeneic HCT, and are often proceeded by a history of acute GVHD.

Two distinct types of GVHD are clinically recognized, acute and chronic. The acute form of the disease usually develops within the first three months after transplantation. The incidence rate of acute GVHD is estimated at 30-50% among patients receiving transplant from HLA-identical sibling donors, and 50-70% in patients receiving HLA-matched unrelated transplants. Severe acute GVHD (grade III-IV) occurs in up to 20% of recipients of related donors and up to 35% of unrelated donors. Severe acute GVHD carries a poor prognosis, with 25% long term survival for grade III and 5% for grade IV.

The skilled person will be able to identify an individual in need for treatment or prevention of GVHD, including by identifying patient groups most likely to receive cell transplants and patients at risk of acute or showing signs of chronic GVHD. For example, chronic GVHD occurs in up to 60% of patients receiving HLA-identical sibling marrow grafts and 70% of patients receiving alternative donor marrow grafts who survive beyond day 100. Symptoms of chronic GVHD usually present between 3 months and 2 years after allogeneic transplantation, and about two thirds develop within the first 12 months. Altogether, only less than 20% of transplanted patients do not develop either acute or chronic GVHD.

Multiple Sclerosis

Multiple sclerosis (MS) is the most common immune-mediated disorder affecting the central nervous system and is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances.

The multiple sclerosis may include active and stable relapsing-remitting multiple sclerosis, primary progressive, secondary progressive and progressive relapsing multiple sclerosis.

Treatment of multiple sclerosis according to the various embodiments of the invention may result in alleviation or amelioration of symptoms, prevention of progression, regression of the condition, or complete recovery. Measurable parameters of successful treatment include one or more, up to all, of Multiple Sclerosis Severity Score or MRI. In specific embodiments, a single treatment is sufficient to cause a depletion of around 10%, 20%, 30%, 40%, 50%, 60% or 70%, or higher up to 80%, 90%, 95% or more, or any range of values between and including these amounts, of one or more of a Th17 or Th22 pathogenic T cell associated with MS, or of a T cell expressing CCR6 from the peripheral blood of the patient. In specific embodiments, at least around 50% depletion is achieved in a single treatment. Subsequent treatment with short chain fatty acids, as herein described, is then useful for preventing the relapse of the MS, or for preventing the re-appearance or repopulation of pathogenic T cell populations associated with MS,

The multiple sclerosis may be diagnosed by standard techniques in the art, familiar to the skilled person. For example, the multiple sclerosis may be diagnosed on the basis of levels of pathogenic T cells, including CCR6 expressing cells, such as lymphocytes and in particular T lymphocytes. A positive diagnosis may be made in subjects based upon the presence of greater than about 18%, greater than about 20% or greater than about 22% CCR6 expressing T cells in the sample, as a percentage of total cells in the sample. Multiple sclerosis may also be diagnosed on the basis of the presence of a about a 1.2 fold or greater increase, such as about a 1.5, 2, 2.5, 3, 3.5, 4, 4.5, 5, 10, 20, 50 or 100 or greater fold increase in CCR6 expressing cells, such as lymphocytes and in particular T lymphocytes, relative to healthy controls.

Progression of multiple sclerosis, which may be in the context of a treatment regime, is monitored on the basis of levels of chemokine receptor expressing cells at different time points. Progression of multiple sclerosis may be indicated in subjects based upon an increase of greater than about 3%, greater than about 4%, greater than about 5%, such as an increase of greater than about 6%, greater than about 7%, greater than about 8%, greater than about 9%, greater than about 10%, greater than about 12%, greater than about 15%, greater than about 20%, greater than about 25%, greater than about 30%, greater than about 35%, greater than about 40%, greater than about 45% or more chemokine receptor expressing cells in the sample, as a percentage of total cells in the sample, compared to a sample taken from the same subject at an earlier time point. In other embodiments, progression of multiple sclerosis is confirmed on the basis of the presence of a about a 1.2 fold or greater increase, such as about a 1.5, 2, 2.5, 3, 3.5, 4, 4.5, 5, 10, 20, 50 or 100 or greater fold increase in chemokine receptor expressing cells, relative to a sample taken from the same subject at an earlier time point.

Multiple sclerosis may be monitored on the basis of levels of pathogenic T cells, including CCR6 expressing cells, such as lymphocytes and in particular T lymphocytes. Progression of the disease, which may be in the context of a treatment regime, may be indicated in subjects based upon the presence of an increase of greater than about 3%, greater than about 4%, greater than about 5%, such as an increase of greater than about 6%, greater than about 7%, greater than about 8%, greater than about 9%, greater than about 10%, greater than about 12%, greater than about 15%, greater than about 20%, greater than about 25%, greater than about 30%, greater than about 35%, greater than about 40%, greater than about 45% or more chemokine receptor expressing cells in the sample, as a percentage of total cells in the sample, compared to a sample taken from the same subject at an earlier time point. In other embodiments, progression of multiple sclerosis is confirmed on the basis of the presence of a about a 1.2 fold or greater increase, such as about a 1.5, 2, 2.5, 3, 3.5, 4, 4.5, 5, 10, 20, 50 or 100 or greater fold increase in pathogenic T cells, including CCR6 expressing cells, such as lymphocytes and in particular T lymphocytes, relative to a sample taken from the same subject at an earlier time point.

Regression or successful treatment may be monitored based upon similar decreases over various time points. For example, regression or successful treatment may be indicated in subjects based upon a decrease of about 3%, such as a decrease of about 4%, about 5%, about 6%, about 7%, about 8%, about 9%, about 10%, about 12%, about 15%, about 20%, about 25%, about 30%, about 35% or more chemokine receptor expressing cells in the sample, as a percentage of total cells in the sample, compared to a sample taken from the same subject at an earlier time point. In other embodiments, regression of multiple sclerosis is confirmed on the basis of the presence of a about a 1.2 fold or greater decrease, such as about a 1.5, 2, 2.5, 3, 3.5, 4, 4.5, 5, 10, 20, 50 or 100 or greater fold decrease in chemokine receptor expressing cells, relative to a sample taken from the same subject at an earlier time point.

Pharmaceutical Formulations

The present invention provides pharmaceutical formulations which enable delivery of an agent that modifies the ratio of effector T cells to T regs or that reduces the number or activity of T cells, in combination with SCFAs in the individual requiring treatment for an autoimmune disease.

The various agents which can be used for depletion or neutralisation of T cells is described earlier in this document.

SCFAs

It will be understood to those skilled in the art, that the pharmaceutical formulations described herein may include SCFAs in the form of free fatty acids, esters or salts or alternatively, as conjugates, such as acetylated or butyrylated starches.

The pharmaceutical formulations described herein may comprise a single species of SOFA or combinations of two or more SCFAs. For example, in performing the methods of the present invention, a person requiring treatment for an autoimmune disease may be administered a single pharmaceutical dosage form comprising a combination of two or more SCFAs. For example, the dosage form may comprise acetic acid and butyric acid, salts or esters thereof. Alternatively, the dosage form may comprise acetic acid and propionic acid, salts or esters thereof, or butyric acid and propionic acid and salts thereof. Yet further, the dosage form may comprise all three of acetic acid, butyric acid and propionic acid, including salts or esters thereof.

In a particularly preferred embodiment, the method of the present invention is performed by administering to an individual in need thereof, a pharmaceutical dosage form comprising a therapeutically effective amount of butyric acid and acetic acid, salts or esters thereof.

The methods of the present invention also contemplate the provision of sequential or simultaneous dosing with one or more pharmaceutical dosage forms comprising a single species of SOFA.

The pharmaceutical compositions of the invention may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The pharmaceutical compositions of the invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the SOFA active ingredients in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatine or acacia, and lubricating agents, for example magnesium stearate, stearic acid or talc.

Delayed Release Oral Dosage Forms

The present inventors have surprisingly found that delivery of a high dosage combination of acetate and butyrate to the lower intestinal tract of a subject, impacts greatly on the development and progression of autoimmune disease, and in particular type 1 diabetes (T1D). The methods of the present invention and the pharmaceutical formulations used in those methods, allow for a very high level of SOFA to be provided in the small or large intestine, including the colon, so as to contribute directly to improvements in gut homeostasis, gut bacterial ecology and Treg numbers and function.

Accordingly, in one embodiment, the present invention relates to a method of treating or preventing an autoimmune disease in an individual in need thereof, the method including:

administering to the individual an agent capable of reducing the activity or viability of a T cell; and

administering to the individual a pharmaceutical dosage form including a therapeutically effective amount of a combination of two or more short chain fatty acids, esters or salts thereof, wherein the short chain fatty acids are selected from the group consisting of butyric acid and acetic acid and propionic acid;

wherein the pharmaceutical dosage form is adapted for release of the short chain fatty acids into the lower gastrointestinal tract of the individual;

thereby treating or preventing the autoimmune disease.

The skilled person will be familiar with methods for producing delayed release oral dosage forms which facilitate delivery of the active agents to a desired region of the gastrointestinal tract. Accordingly, in one embodiment, the present invention relates to oral dosage forms comprising two or more SCFAs, and a pharmaceutically effective excipient, wherein the dosage form is adapted for release of the SCFAs into the large intestine.

The term “delayed release,” as used herein, refers to a delivery of SCFAs which is achieved by formulating the pharmaceutical composition comprising the SCFAs so that their release will be accomplished at some generally predictable location in the lower GI tract more distal to that which would have been accomplished had there been no alteration in the delivery of the SCFAs.

The term “gastrointestinal tract” or “GI tract,” as used herein, relates to the alimentary canal, i.e., the musculo-membranous tube about thirty feet in length, extending from the mouth to the anus. The term “upper gastrointestinal tract,” as used herein, means the buccal cavity, the pharynx, the esophagus, and the stomach. The term “lower gastrointestinal tract,” as used herein, means the small intestine and the large intestine.

The term “small intestine,” as used herein, means the part of the lower gastrointestinal tract consisting of the duodenum, the jejunum, and the ileum, i.e., that portion of the intestinal tract just distal to the duodenal sphincter of the fundus of the stomach and proximal to the large intestine.

The term “large intestine,” as used herein, means the part of the lower gastrointestinal tract just distal to the small intestine, beginning with the cecum, including the ascending colon, the transverse colon, the descending colon, the sigmoid colon, and the rectum.

In certain embodiments of the present invention, it may be desirable to achieve delivery of the SCFAs (either as free acids, salts or esterified acids) to the small intestine or a particular segment thereof (e.g., the duodenum, jejunum or ileum). In still other instances, it may be desirable to deliver the SCFAs in a bolus amount to the small intestine.

In certain embodiments of the present invention, it may be desirable to achieve delivery of the SCFAs (either as free acids, salts or esterified acids) to the large intestine or a particular segment thereof (e.g., the ascending colon). In still other instances, it may be desirable to deliver the SCFAs in a bolus amount to the large intestine.

In one embodiment, the oral dosage form comprises an enteric coating which is resistant to degradation in the stomach, but which dissolves once the dosage form exits the stomach and enters the small intestine. In an alternative embodiment, the oral dosage form comprises an enteric coating which is resistant to degradation in the stomach and small intestine but dissolves once the dosage form arrives in the large intestine. The skilled person will be familiar with the use of enteric coating materials to control release of the active ingredient contained in the pharmaceutical dosage form such that the active ingredient is released in a specified location in the gastrointestinal tract.

The skilled person will appreciate that the ultimate site of and/or the rate of delivery in the small or large intestine can be satisfactorily controlled by one skilled in the art, by manipulating any one or more of the following:

(a) the type of coating, the type and level of excipients added to the coating and the concomitant desirable thickness and permeability (swelling properties) of the coating;

(b) the time dependent conditions of the coating itself and/or within the coated tablet, particle, bead, or granule;

(c) the pH dependent conditions of the coating itself and/or within the coated tablet, particle, bead, or granule;

(d) the dissolution rate of the coating;

A human or other mammal suffering from and requiring treatment for an autoimmune disease, can in certain embodiments of the present invention, be successfully treated by the delivery of SCFAs to the large intestine of said human or other mammal. The dosage forms described herein effect a release to the large intestine, and prohibit the undesired release of the SCFAs in the mouth, pharynx, esophagus, stomach, and/or small intestine, thereby preventing the degradation of the SCFAs before they release their intended site in the gastrointestinal tract.

Various means for targeting release of the SCFAs in the small or large intestine, including the colon are suitable for use in the present invention. Non-limiting examples of means for delivery to the large intestine include pH triggered delivery systems and time dependent delivery systems.

One embodiment of the present invention involves coating (or otherwise encapsulating) the SCFAs with a substance which is not broken down, by the gastrointestinal fluids to release the SCFAs until a specific desired point in the intestinal tract is reached. In one embodiment, delayed release of the pharmaceutical composition is achieved by coating the tablet, capsule, particles, or granules, of the SCFAs with a substance which is pH dependent, i.e, broken down or dissolves at a pH which is generally present in the large intestine, but not present in the upper gastrointestinal tract (i.e., the mouth, buccal cavity, pharynx, esophagus, or stomach) or lower GI tract.

One embodiment of the present invention is delivered to the small or large intestine utilizing a pH dependent enteric coating material made from a partly methyl esterified methacrylic acid polymer. The oral dosage form can be in the form of an enteric coated compressed tablet made of granules or particles of active ingredient. Any enteric coating which is insoluble at a pH below 5.0 (i.e., that generally found in the mouth, pharynx, esophagus, stomach), but soluble between about pH 5.5 and about pH 7.5 (i.e., that present in the small and large intestine) can be used in the practice of the present invention. For example, when it is desired to effect delivery of the SCFAs to the large intestine, any enteric coating is suitable which is wholly- or partially-insoluble at a pH below 6.5 and soluble above pH 6.5.

The skilled person will appreciate that the pH varies along the digestive tract and will be able to determine a suitable enteric coating to ensure that the dosage form disintegrates and the active ingredients are released at an appropriate or desired location in the gastrointestinal tract.

Methacrylic acid copolymers which are suitable for use in coating the oral dosage forms and/or the granules, particles, or beads of active ingredient which can be employed in the method of treatment described herein, either alone or in combination with other coatings, are anionic carboxylic polymers. It is particularly preferred that the polymers are acrylic polymers, most preferably partly methyl-esterified methacrylic acid polymers, in which the ratio of anionic free carboxyl groups to ester groups is about 1:1.

A particularly suitable methacrylic acid copolymer is Eudragit L®, particularly Eudragit L-30-D® and Eudragit 100-55®, manufactured by Rohm Pharma GmbH, Weiterstadt, West Germany. In Eudragit L-30-D®, the ratio of free carboxyl groups to ester groups is approximately 1:1. Further, said copolymer is known to be insoluble in gastrointestinal fluids having a pH below 5.5, generally 1.5-5.5, i.e., that generally present in the fluid of upper gastrointestinal tract, but readily soluble at pH above 5.5, i.e., that generally present in the fluid of the lower gastrointestinal tract. Such copolymers are useful for enteric coatings intended to facilitate release of the active ingredients into the small intestine.

Alternative copolymers are Eudragit S® and Eudragit FS30D®, manufactured by Rohm Pharma GmbH and Co. KG, Darmstadt, Germany. Eudragit differs from Eudragit L 30 D-55®, only insofar as the ratio of free carboxyl groups to ester groups is approximately 1:2. Eudragit S® is also, like Eudragit L 30 D-55®, substantially insoluble at pH below 5.5, but unlike Eudragit L 30 D-55®, is poorly soluble in GI fluids having a pH of 5.5-7.0, such as that present in small intestinal fluids. Eudragit S® is soluble at pH 7.0 and above, i.e., that generally present in the terminal ileum and colon.

Eudragit S® can also be used alone as a coating which would provide delivery of the SOFA ingredients beginning primarily at the large intestine (more distal than the terminal ileum) via a delayed-release mechanism. In addition, Eudragit S®, being poorly soluble in intestinal fluids below pH 7.0, could be used in combination with Eudragit L 30 D-55®, soluble in intestinal fluids above pH 5.5, in order to effect a delayed release composition which could be formulated to deliver the active ingredient at various segments of the intestinal tract; the more Eudragit L 30 D-55® used, the more proximal release and delivery begins and the more Eudragit S® used, the more distal release and delivery begins.

The coating can, and usually will, contain a plasticizer and possibly other coating excipients such as coloring agents, surfactant, talc, and/or magnesium stearate, many of which are well known in the coating art. In particular, anionic carboxylic acrylic polymers usually will contain 10-25% by weight of a plasticizer, especially triethyl citrate, tributyl citrate, acteyltriethyl citrate, dibutyl phthalate, diethyl phtbalate, polyethylene glycol, acetylated monoglycerides, propylene glycol, and triacetin.

Conventional coating techniques such as fluid-bed or pan coating are employed to apply the coating. Coating thickness must be sufficient to ensure that the oral dosage form remains essentially intact until the desired site of delivery in the small intestine is reached.

The oral dosage form may be in the form of a coated compressed tablet which contains particles or granules of the SCFAs, or of a soft or hard capsule (e.g., gelatine, starch, or hydroxypropylmethylcellulose), coated or uncoated, which contains beads or particles of the SCFAs, which themselves are enterically coated. In an embodiment of the invention the tablets are compressed and the tablet is enteric coated.

Time Dependent Delivery Systems and Bacterial Enzyme Triggered Systems

In another embodiment of the invention, delivery of the SCFAs to the large intestine is achieved through the use of a time dependent delivery system. Given established transit times after gastric emptying, SCFA release (either as free acid or esterified acids) can be targeted to the various segments of the large intestine.

Approaches to time dependent delivery systems suitable for use in the present invention include, but are not limited to, such devices as the Pulsincap™ (Scherer DDS, Strathclyde, U.K.), the Time Clock™ (Zambon Group, Milan, Italy), and SyncroDose™ (Penwest, Patterson, N.Y.), as well as various coatings which degrade over time to release tablet contents such as hydroxypropylmethylcellulose, hydroxypropylcellulose, or any suitable hydrogel.

In one embodiment of the invention, delivery of the SCFAs to the large intestine is achieved through the use of a bacterial enzyme triggered system. Oral dosage forms from which drug release is triggered by the action of bacterial enzymes in the colon are known in the art. Various approaches to bacterially-triggered delivery systems suitable for use in the present invention include disulfide polymers, glycosidic prodrugs, and polysaccharides as matrices/coating agents (Watts & Ilium, 1997). Further approaches to bacterially-triggered delivery systems suitable for use are disclosed in Katsuma et al., 2004). In one embodiment of the invention, the colon-targeted delivery system CODES™ (Yamanouchi Pharma Technologies, Norman, Okla.) is used to deliver the SCFAs to the colon. This system comprises a tablet core containing SCFAs, and a saccharide, which tablet core is coated with an acid soluble material, such as Eudragit E®, and then coated with an enteric coating, such as Eudragit L®. The enteric coating protects the dosage form from degradation in the stomach, and is subsequently dissolved in the small intestine following gastric emptying. The acid-soluble coating protects against degradation as the dosage form travels through the small intestine. When the dosage form reaches the large intestine, local microflora ferment the saccharide (e.g., lactulose) in the tablet core into short chain fatty acids (such as isobutyrate, butyrate, isovalerate, valerate, isocaproate and caproate) which then dissolve the acid-soluble coating to release the core tablet contents in the colon.

Accordingly, the use of the CODES system provides yet a further means of providing an increased amount of butyric acid to the colon of an individual in need thereof. Thus, in yet a further embodiment, the present invention contemplates the provision of SCFAs to the colon of an individual, comprising administering to the individual a pharmaceutical dosage form including a therapeutically effective amount of acetic acid, esters or salts thereof, wherein the dosage form comprises a tablet core, a saccharide, an inner enteric coating in the form of an acid-soluble enteric coating (such as Eugradit E) and an outer enteric coating acid-resistant enteric coating (such as Eudragit L).

In a further embodiment, the dosage form also comprises a therapeutically effective amount of butyric acid, esters or salts thereof in the tablet core.

The skilled person will be familiar with alternative methods which may be employed for colon-targeted delivery of SCFAs including pressure dependent systems, CODES™ technology, microsponges, pectin and galactomannan coating, microbially triggered osmotic systems and lectins.

Formulations for oral use may also be presented as hard gelatin capsules wherein the SCFAs are mixed with an inert solid diluent, for example, calcium carbonate, calcium phosphate or kaolin, or as soft gelatin capsules wherein the SCFAs are mixed with water or an oil medium, for example peanut oil, liquid paraffin, or olive oil.

Aqueous suspensions may contain the SCFAs in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the SCFAs in a vegetable oil, for example arachis oil, olive oil, sesame oil or coconut oil, or in a mineral oil such as liquid paraffin. The oily suspensions may contain a thickening agent, for example beeswax, hard paraffin or acetyl alcohol. Sweetening agents such as those set forth above, and flavouring agents may be added to provide a palatable oral preparation. These may be preserved by the addition of an anti-oxidant such as ascorbic acid.

Dispersible powders and granules suitable for preparation of an aqueous suspension by the addition of water provide the SCFAs in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxy ethylene sorbitan monooleate. The emulsions may also contain sweetening and flavouring agents.

Syrups and elixirs may be formulated with sweetening agents, for example glycerol, propylene glycol, sorbitol or sucrose. They may also contain a demulcent, a preservative and flavoring and coloring agents.

Rectal Suppositories

The method of the present invention also contemplates the provision of SCFAs in the large intestine via a pharmaceutical dosage form which is provided as a rectal suppository. Accordingly, in a particular embodiment, the pharmaceutical compositions of the invention are formulated as suppositories for rectal administration of the SCFAs. These formulations can be prepared by mixing the SCFAs with a suitable non-irritating excipient which is solid at ordinary temperatures but liquid at the rectal temperature and will therefore melt in the rectum to release the drug. Such materials include cocoa butter and polyethylene glycols. Rectal administration may be used to eliminate entero-hepatic first pass effect in the gastro-intestinal tract related to oral administration of active agents. In yet a further embodiment, the rectal suppositories may include the SCFAs provided as esterified modified starches, wherein upon release of the modified starch in the rectum, the starch becomes available to the resident microbiota for digestion and consequent release of the SCFAs as metabolites of digestion.

Injectable Formulations

The present invention also involves the use of injectable pharmaceutical formulations for systemic delivery of the SCFAs. For example, the SCFAs may be directly injected into the bloodstream of the individual for whom treatment or prevention of an autoimmune disease is required. The injection may be adapted for intravenous or intraarterial injection. In an alternative embodiment, the injectable formulation may be adapted for subcutaneous injection, so as to facilitate either local administration, or a delayed release into the bloodstream.

The pharmaceutical compositions of invention may be in the form of a sterile injectable aqueous or oleagenous suspension. This suspension may be formulated according to the known art using those suitable dispersing or wetting agents and suspending agents which have been mentioned above. The pharmaceutical compositions may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical compositions of the invention may conveniently be presented in dosage unit form and may be prepared by any of the methods well known in the art of pharmacy. All methods include the step of bringing the SCFAs together with the pharmaceutically acceptable excipients which constitutes one or more accessory ingredients. In general, the pharmaceutical compositions are prepared by uniformly and intimately bringing the SCFAs into association with a liquid carrier or a finely divided solid carrier or both, and then, if necessary, shaping the product into the desired formulation.

The pharmaceutical compositions of the invention, may also be formulated in liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolisable lipid capable of forming liposomes can be used. The liposome formulation may contain stabilisers, preservatives, excipients and the like. The preferred lipids are the phospholipids and phosphatidyl cholines, both natural and synthetic. Methods to form liposomes are known in the art.

The pharmaceutical compositions of the invention, may be included in a container, pack, or dispenser together with instructions for administration. The SCFAs, and optionally additional active agent, of the pharmaceutical composition may be provided as separated components in the container, pack, or dispenser, to be taken separately or together at the same or different time in a use or method of the invention described herein.

The term “pharmaceutically acceptable” as used herein means the carrier, diluent or excipient is not deleterious to the recipient thereof.

Unless otherwise indicated herein, the terms “composition” and “formulation” are used interchangeably.

The terms “administration of” and or “administering” should be understood to mean providing to an individual in need of treatment.

Dietary Agent

Acylated high-amylose starches are known. For example, such starches are described in U.S. Pat. No. 5,840,860, and Annison et al., 2003 the entire contents of which are herein incorporated by reference (with particular reference to Examples 1, 3, 5, 6 and 8). Specifically, acetylated high-amylose maize starch (HAMSA), butyrylated high-amylose maize starch (HAMSB) and propionate high-amylose maize starch (HAMSP) are known, as is high-amylose maize starch which does not comprise esterified fatty acid (HAMS). The use of these acylated starches individually is known, however, before now, utilising combinations of these starches has not previously been contemplated, particularly in combination with a treatment (or pre-treatment) which is aimed at depleting effector and memory T cells.

The present inventors have surprisingly found that delivery of a high dosage combination of acetate and butyrate through diet is a simple approach that impacts greatly on the development and progression of autoimmune disease, following pan-T cells targeting and in particular type 1 diabetes (T1D). Diet and bacterial metabolites represents a highly promising alternative to pharmaceutical approaches, to prevent or treat T1D and other autoimmune diseases.

The methods of the present invention allow for a very high level of SOFA to be released in the lower colon, and significantly higher levels than those obtained through intake of dietary fibre alone. Specifically, because the acylated starches described herein are resistant to degradation in the small intestine of the individual, and have higher amounts of fatty acid than starches found in normal diets, the dietary agents and diets described herein enable the provision of significantly higher doses of short chain fatty acids in the large intestine of an individual which can be administered or taken by the individual in a convenient and safe form.

Exemplary methods for preparing dietary agents for use in the method of the present invention are further described herein.

Dietary metabolites operate at many levels to correct defects in gut or immune homeostasis, limit number of autoreactive T cells, and prevent disease. One important feature of the present invention is that dietary delivery of high amounts of acetate, a natural product, was shown to achieve changes in the B cell molecular profile in the whole animal. Further, butyrate was found to protect against T1D through a Treg associated pathway, distinctive from that described for acetate. Accordingly, the particular combination of a high acetate and butyrate treatment represents an exciting and simple means for manipulating the cells of the immune system, not just cells present in the colon.

The intestinal microbiota responds rapidly to changes in diet and prolonged use of certain diets permits depletion of stressed and uncompetitive bacteria and emergence of new faster growing strains due to adaptive mutations. In summary, the present inventors have demonstrated that the approach to using dietary metabolites represent a novel and effect means for disruption of autoimmune pathogenesis.

Accordingly, the present invention further provides a dietary agent for delivery of two or more short chain fatty acids into the large intestine of an individual, the agent including a carrier covalently bonded to a plurality of short chain fatty acids, wherein the short chain fatty acids include two or more of acetic acid, butyric acid and propionic acid, and wherein the short chain fatty acids are bound to the carrier by a bond that is hydrolysable in the colon of an individual, to give free fatty acid.

For example, in one embodiment, each molecule of carrier includes an acetic acid and a butyric acid moiety. In an alternative embodiment, each molecule of carrier includes and acetic acid and a propionic acid moiety, or a propionic acid and a butyric acid moiety. In a further embodiment, each molecule of carrier includes at least one acetic acid, at least one butyric acid and at least one propionic acid molecule. In yet further embodiments, each molecule of carrier includes a plurality of SOFA moieties wherein the SCFAs are a combination of two or more of acetic acid, butyric acid or propionic acid.

In a preferred embodiment, the carrier molecule is a carbohydrate, although it will be appreciated by those with skill in the art, that other carriers may be used. The carbohydrate can be a pectin, gum, mucilage, cellulose, hemicellulose, inulin or oligosaccharide. Preferably the carbohydrate is a starch.

In yet another preferred embodiment, the dietary agent of the present invention includes a starch molecule acylated with two different SCFAs. For example, the starch may be acylated with a plurality of both butyric acid and acetic acid moieties. In a further embodiment, the starch is acylated with a plurality of butyric, acetic and propionic acid moieties.

The present invention also contemplates various carrier molecules having various degrees of substitution. For example, where the carrier is a starch molecule, the invention contemplates degrees of substitution ranging from 0.05 to 1.0, preferably 0.1 to 0.8 and more preferably 0.5. A degree of substitution of 0.5 means that on average throughout the starch molecule, there is one short chain fatty acid moiety per 2 glucose molecules. The skilled person will appreciate that the presence of short chain fatty acid moieties will not necessarily be uniform along the length of the starch molecule, but represents the number of moieties on average. The skilled person will also appreciate that where two short chain fatty acid moieties are present on a single molecule of starch, a degree of substitution of 0.5 is indicative of roughly 1 short chain fatty acid of one type per 4 molecules, assuming there are equal amounts of each short chain fatty acid moiety on the starch molecule.

Combination Diet

It will be appreciated that the present invention also relates to the provision of at least two dietary agents to an individual, for use in the methods of treatment and prevention described herein. For example, the dietary agents may each be for delivery of a single species of short chain fatty acid to the large intestine of an individual, wherein each agent includes a carrier, and each molecule of carrier is covalently bonded to a short chain fatty acid molecule by a bond that is hydrolysable in the colon of an individual, to give free fatty acid. The degree of substitution of each fatty acid on the carrier is between 0.1 and 0.5, preferably 0.15 to 0.20.

Accordingly, the present invention also relates to a combination diet, the diet comprising two or more dietary agents described above, for providing in the diet of an individual in need thereof, two or more short chain fatty acids.

Preferably, the combination diet comprises a combination of acetylated starch and butyrylated starch, each prepared as described in U.S. Pat. No. 5,840,860. In another embodiment, the combination diet comprises a combination of acetylated starch and propionylated starch. In yet a further embodiment, the combination diet comprises a combination of butyrylated starch and propionylated starch.

Methods for preparing dietary agents comprising a single species of short chain fatty acid are known, and described in U.S. Pat. No. 5,840,860, herein incorporated by reference and further described in the Examples.

Dosage

It will be well within the purview of the person skilled in the art to determine an appropriate dosage of a T-cell depleting, or modulating treatment as described herein for the purpose of reducing the number, or activity of a population of T cells, including various subpopulations of T cells.

The present methods contemplate the provision of a range of dosages of short chain fatty acids. It will be appreciated that the dose may vary depending on the mode of administration of the SOFA, the form in which it is provided (e.g., as an oral dosage form, injection or dietary agent) and the intended site of action of the short chain fatty acids.

Preferably, where the method involves the administration of an oral dosage form for delivery of the combination of short chain fatty acids into the large intestine of an individual, the dose is approximately 0.01 mg/kg to 100 mg/kg per day, preferably 0.1 mg/kg to 100 mg/kg per day, more preferably 1 mg/kg to 50 mg/kg. In a most preferred embodiment, the daily dose of any one of acetic acid, butyric acid or propionic acid is 2 mg/kg to 10 mg/kg, including 3, 4, 5, 6, 7, 8, and 9 mg/kg.

In circumstances where the method of the invention relates to the use of a dietary agent or combination diet for delivery of the combination of short chain fatty acids into the large intestine, the skilled person will appreciate that the amount of agent or diet to be consumed will vary depending on the composition of the diet and the proportion of each short chain fatty acid incorporated into the carrier molecules included in the dietary agent.

In one embodiment, where the method relates to the provision of a dietary agent comprising a combination of two short chain fatty acids bound to a single carrier molecule, and the carrier is a starch having a degree of substitution of 0.4 (i.e., 1 short chain fatty acid approximately every 2.5 molecules), the dosage will be approximately 1 g of starch to 40 g of starch per day for a 50 kg individual, preferably 1 g of starch per day to 10 g of starch per day (i.e. 0.02 g to 0.2 g/kg per day). More preferably, the dose will be 2 g to 8 g per day (0.04 g/kg to 0.16 g/kg per day). More preferably the dose if approximately 3.75 g of the starch molecule per day for a 50 kg individual (or 0.075 g/kg/day). This corresponds to approximately 250 mg to 300 mg of each of the short chain fatty acids, assuming there are equal proportions of these in the starch molecule.

Alternatively, the method may also involve the administration of a combination diet comprised of two forms of acylated starch combined to provide two short chain fatty acids. For example, a starch having been modified with acetic acid and having a degree of substitution of 0.2 (i.e., 1 acetic acid per 5 glucose molecules on average) may be used at a dose of 0.04 g/kg to 0.16 g/kg per day, more preferably 0.05 g/kg to 0.1 g/kg per day and yet more preferably 0.075 g/kg/day.

Administration

The skilled person will be familiar with appropriate administration for the agent capable of reducing the viability of a pathogenic immune cell (e.g. T cell) described herein. It will be appreciated that typically, the T cell-targeting agent will be administered separately to the SOFA treatment.

In one embodiment, the SCFAs are provided in the individual for release in the large intestine of the individual, for contacting the cells of the large intestine with the SCFAs. This can be accomplished by a number of means, including the use of an enteric coated dosage form for oral administration, which is formulated for release of the SCFAs in the colon of the individual. Alternatively, the SCFAs can be provided in a pharmaceutical dosage form for rectal administration.

The pharmaceutical compositions of the invention may be administered by any suitable means, for example, orally, such as in the form of tablets, capsules, granules or powders; sublingually; buccally; parenterally, such as by subcutaneous, intravenous, intramuscular, intraperitoneal or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories or enemas; in dosage unit formulations containing non-toxic, pharmaceutically acceptable vehicles or diluents. They may, for example, be administered in a form suitable for immediate release or extended release, for example, by the use of devices such as subcutaneous implants, encapsulated spheroids or osmotic pumps.

In an alternative embodiment, the methods of the present invention involve the administration of SCFAs via a dietary agent or combination diet as herein described.

In yet further embodiments, the present invention contemplates the provision of SCFAs by more than one means of administration such that a first species of SOFA may be administered by one means of administration, and a second species of SOFA administered by an alternative means. For example, in one embodiment, the present invention may include providing a first SOFA by administration of an oral dosage form and a second SOFA by intravenous injection of a dosage form adapted for intravenous injection. This may be particularly useful in the administration of SCFAs which are susceptible of first pass metabolism, such as butyric acid. However, the skilled person will appreciate that any number of combinations may be utilised for providing the SCFAs to the individual in need (i.e., combinations of pharmaceutical dosage forms, a combination of a dietary agent and a pharmaceutical dosage form or a combination of dietary agents).

Accordingly, in one embodiment, the present invention includes a method of preventing or treating an autoimmune disease in an individual in need thereof, comprising administering to the individual, a first pharmaceutical dosage form comprising a first species of SOFA, and a second pharmaceutical dosage form comprising a second species of SOFA, wherein the first pharmaceutical dosage form is adapted for parenteral injection and the second pharmaceutical dosage form is adapted for oral administration.

In a preferred embodiment, the present invention includes a method of preventing or treating an autoimmune disease in an individual in need thereof, comprising administering to the individual, a first pharmaceutical dosage form comprising butyric acid, esters or salts thereof and a second pharmaceutical dosage form comprising acetic acid, esters or salts thereof, wherein the first pharmaceutical dosage form is adapted for parenteral injection and the second pharmaceutical dosage form is adapted for oral administration.

In yet an alternative embodiment, the invention provides a method of preventing or treating an autoimmune disease in an individual in need thereof, comprising administering to the individual, a first pharmaceutical dosage form comprising acetic acid, esters or salts thereof and a second pharmaceutical dosage form comprising a butyric acid, esters or salts thereof, wherein the first pharmaceutical dosage form is adapted for parenteral injection and the second pharmaceutical dosage form is adapted for oral administration.

Alternatively, the invention provides a method of preventing or treating an autoimmune disease in an individual in need thereof, comprising administering to the individual, a pharmaceutical dosage form comprising acetic acid, esters or salts thereof and a dietary agent comprising a butyric acid, esters or salts thereof. The pharmaceutical dosage form may be adapted for parenteral injection (including intravenous or subcutaneous injection), oral administration or as a rectal suppository.

Combination Therapy

The present invention contemplates the use of the pharmaceutical formulations, dietary agents or combination diet described herein, in combination with other methods for providing high levels of SCFAs to the large intestine of an individual requiring treatment or prevention of an autoimmune disease.

For example, the methods of the present invention include the prior, simultaneous or sequential provision to an individual of one or more agents including prebiotics or probiotics, which may be used to alter the composition of the microbiome in the intestine of the individual. The skilled person will appreciate that such pre- or pro-biotics include genetically modified bacteria or non-genetically modified bacteria.

Further, the present invention also contemplates the use of the pharmaceutical formulations, dietary agents or combination diet described herein, in combination with other methods for preventing or treating autoimmune disease.

In the context of treating type I diabetes, the present invention contemplates a combination therapy including administration of insulin injections and administration of SCFAs according to any of the methods described herein. For example, in one embodiment, the present invention contemplates administering to an individual in need thereof, an oral dosage form adapted for delayed release of two or more species of SOFA into the large intestine of an individual diagnosed with type I diabetes, wherein the individual is further receiving subcutaneous injections of insulin.

In a preferred embodiment, the invention relates to a method of treating type I diabetes in an individual in need thereof, comprising administering to the individual:

an oral dosage form comprising a therapeutically effective amount of acetic acid and butyric acid, salts or esters thereof,

wherein the oral dosage form is adapted for delayed release of the acetic acid and butyric acid, salts or esters thereof, into the large intestine of the individual; and

a therapeutically effective amount of insulin

thereby treating type I diabetes in the individual.

### EXAMPLES

### Example 1: Animal studies

Experimental materials and methods

Animals and Models

The NOD/Lt (NOD), C57BL/6 and NOD.8.3 mice were derived from Monash Animal Research Platform, Melbourne Australia. Gpr43−/− mice (Maslowski, et al., (2009) Nature 461, 1282-1286) and the MyD88−/− mice (obtained from Shizuo Akira), both on a C57BL/6 background were backcrossed >10 times to the NOD background. GF NOD mice were derived from Germ Free Unit (Walter and Eliza Hall Institute of Medical Research). NOD.FoxP3-GFP mice (NOD/ShiLt-Tg(FoxP3-EGFP/cre)1cJbs/J, obtained from The Jackson Laboratory, USA).

To standardise microbiota, prior to beginning diets mice from multiple litters were mixed and randomly allocated to groups. The purified diets used were based on a balanced modification of the AIN93-G diet as described previously (Bajka et al., Br J Nutr. (2006) 96(2):276-82). Mice were fed for 3-5 weeks starting at 3, 5 or 10 weeks of age. SCFAs in faeces, blood and caecal content were analysed as previously described in (Bajka et al., 2006, supra). Diabetes was monitored as previously described (Marino et al., (2009) Diabetes 58, 1568-1577).

Animal Diets

MCD Diet to Induce NASH

The MCD diet was as described in the prior art (low choline and low methionine; see for example Machado et al., 2015, PLoS One; 10(5): e0127991).

High butyrate and high acetate-yielding diet

Control (LAMS), and acetylated (HAMSA), proprionylated (HAMSP) and butyrylated (HAMSB) starch diets contained the following ingredients:

components as g/kg

| | | 15% | 15% | 15% |
|---|---|---|---|---|
| Ingredient | LAMS | HAMSA | HAMSP | HAMSB |
| Maize starch-3401C | 529.5 | 379.5 | 379.5 | 379.5 |
| HAMSA | 0 | 150 | 0 | 0 |
| HAMSP | 0 | 0 | 150 | 0 |
| HAMSB | 0 | 0 | 0 | 150 |
| Casein | 200 | 200 | 200 | 200 |
| Sucrose | 100 | 100 | 100 | 100 |
| Sunflower Seed Oil | 70 | 70 | 70 | 70 |
| alpha cellulose | 50 | 50 | 50 | 50 |
| Mineral Mix AIN 93G | 35 | 35 | 35 | 35 |
| Vitamin Mix AIN 93VX | 10 | 10 | 10 | 10 |
| L-Cystine | 3 | 3 | 3 | 3 |
| Choline bitartrate | 2.5 | 2.5 | 2.5 | 2.5 |
| Total | 1000 | 1000 | 1000 | 1000 |

Blood Glucose Levels

Blood glucose levels were assessed using conventional methods. A blood glucose level of >12.0 Mm/I on two consecutive readings was scored as indicative of diabetes.

Histopathology

Pancreatic and liver tissue was processed and stained using standard procedures. For insulitis scoring, pancreata sections were taken at 3 concentrations (100 μm apart). At least 100 islets were scored from 5 to 15 mice. Islets were graded according to the following system: Grade 0—no indication of insulitis, Grade 1—<25% infiltration, Grade 2—25-50% infiltration, Grade 3—50-75% infiltration, and Grade 4—>75% infiltration.

Steatosis was scored and the severity was graded, based on the percentage of the total area affected, into the following categories: 0 (<5%), 1 (5-33%), 2 (34-66%) and 3 (>66%). Inflammation was evaluated by counting the number of inflammatory foci per field using a 100× magnification.

Statistical Analysis

Statistical significance for comparing two independent groups was determined by calculating P-values using non-parametric Mann-Whitney U test (GraphPad Prism software, La Jolla, Calif., USA). For all diet data, the following comparisons were made: (1) NP vs. HAMS, (2) HAMS vs. HAMSA, (3) HAMS vs. HAMSB, (4) NP vs HAMSA, (5) NP vs HAMSB. Diabetes incidence studies were graphed as Kaplan-Meier survival plots and analysed using the Mantel-Cox log-rank test with 2-degrees of freedom. ****P<0.0001, ***P<0.001, **P<0.01, *P<0.05.

Results

Combination Therapy with an Anti-CD3 Antibody Followed by High Acetate and Butyrate-Yielding Diet Protects Against T1D

Diabetic NOD8.3 mice were injected with anti-CD3 antibody (anti-CD3 mAb (hamster anti-mouse clone 145-2C11, Bio X Cell) in order to deplete effector T cells. One week later, mice were commenced on a high acetate and butyrate diet (15% HAMSA/15% HAMSB) as detailed above. Three weeks later, mice receiving the diet were protected from developing diabetes as demonstrated by a sustained, reduced blood glucose level compared to the levels observed prior to T cell depletion.

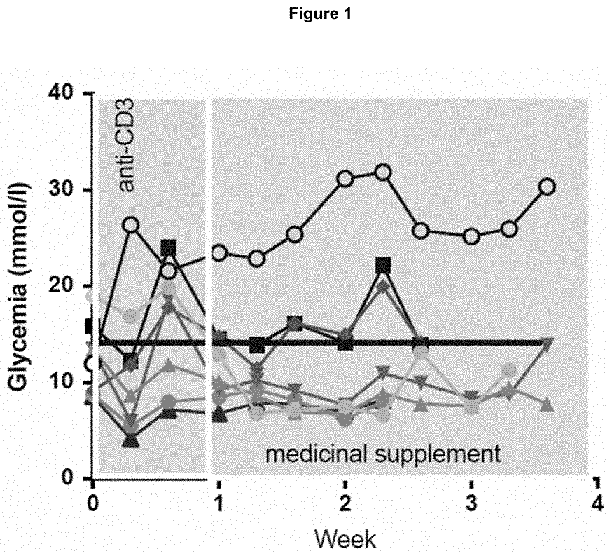

These results (shown in FIG. 1) indicate that combination therapy with a pan T cell depleting agent (such as an anti-CD3 antibody) followed by a diet yielding high levels of acetate and butyrate in the colon is useful for reversing symptoms of diabetes, and providing long-term protection from relapse.

Treatment of NASH

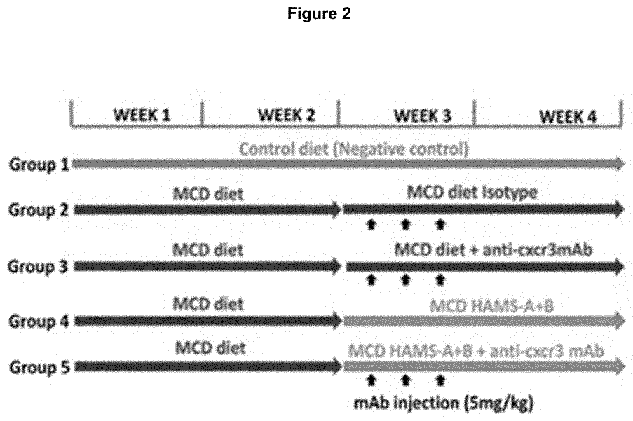

FIG. 2 provides a schematic of the interventions over a 4 weeks period designed to assess the efficacy of various approaches for treating or preventing NASH.

Briefly, mice in Group1 received a control diet. Mice in Group 2 received the MCD diet which is used to induce the onset of NASH. After 2 weeks of being on the MCD diet, mice then received an isotype antibody in addition to continuing the MCD diet.

Mice in Group 3 were placed on the MCD diet for a 2 week period followed by the MCD diet in conjunction with a CXCR3 depleting antibody for the remaining 2 weeks.

Mice in Group 4 received the MCD diet for the first 2 weeks, then a combination of the MCD diet and 15% HAMSA/15% HAMSB diet as shown above, for the remaining 2 weeks.

Mice in Group 5 received the MCD diet for the first 2 weeks, followed by a combination of the MCD diet, the 15% HAMSA/15% HAMSB diet for the following 2 weeks, including 3 injections of the CXCR3 depleting antibody (3×5 mg/kg injections of antibody).

Mice in Group 5 received the MCD diet for the first 2 weeks, follows by a combination of the MCD diet and the LAMS (control starch diet) for the remaining 2 weeks.

Monotherapy with a CXCR3 Depleting Antibody Protects Against NASH

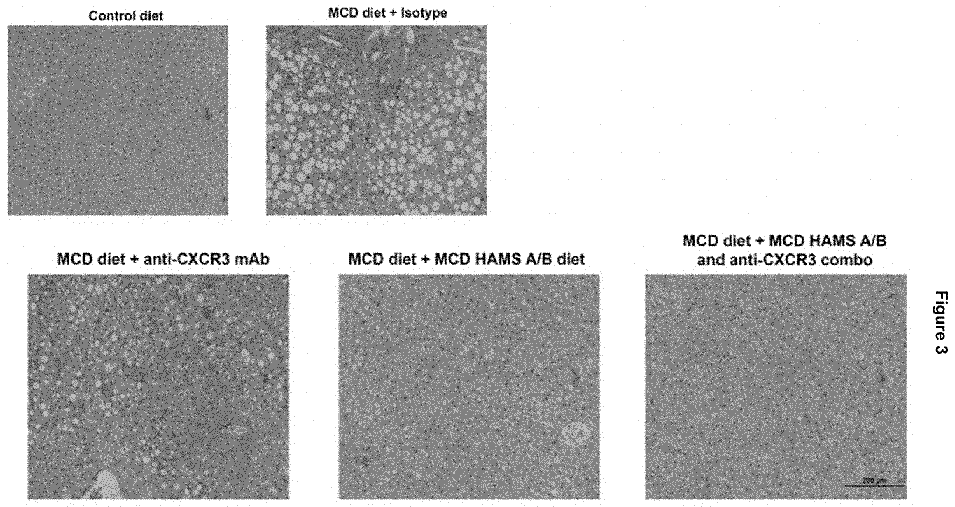

As shown in FIG. 3, the livers of mice in Group 2 has significant fatty deposits at the conclusion of the 4 week trial as compared to mice on the control (normal) diet (Group 1).

The livers of mice in Group 3, who received a CXCR3 depleting antibody treatment in conjunction with the NASH-inducing MCD diet, were significantly protected from the development of fatty deposits. While some fatty deposits are evident in FIG. 3, these are significantly smaller in size and reduced in proportion compared to the mice in Group 3.

These results show that depletion of CXCR3 positive T cells using a CXCR3 depleting antibody alone, is useful for reducing the severity and extent of fatty liver in NASH.

Monotherapy by Increasing SCFAs by High Acetate- and Butyrate-Yielding Diets Protects Against NASH

Mice in Group 4, who received a high acetate and butyrate-yielding diet 2 weeks after commencement of the MCD diet, were also significantly protected from NASH. Livers from mice in Group 4 had significantly smaller fatty liver deposits than mice in either Groups 3 and 2.

These results show that a diet high in acetate and butyrate delivered to the colon alone, is sufficient to provide significant protection from NASH.

Combination Therapy with a CXCR3 Depleting Antibody in Conjunction with a High Acetate and Butyrate-Yielding Diet Protects Against NASH

Mice in Group 5 received the NASH-induced MCD diet for 2 weeks, followed by a combination of the MCD diet, a high acetate and butyrate-yielding diet and a CXCR3-depleting antibody. The livers from mice in Group 5 had no signs of fatty liver deposits, and a similar histological profile to the livers from mice in Group 1.

These results indicate that the combination of a CXCR3 depleting treatment, coupled with a diet high in acetate and butyrate delivered to the colon is useful for preventing the onset of NASH.

### Example 2: Preparation of Acylated Starches Containing Two or More Fatty Acids (Large Scale)

Method:
1) DMSO (24 L) was heated to above 80° C. with an immersion heater in a metal vessel under constant stirring.
2) The heater was removed and maize starch (440 g) was added slowly to the stirring DMSO through a domestic sieve to ensure uniform dispersement (to avoid clumping). The mixture was stirred constantly for 1 h by which time all the starch had dissolved to leave a clear, viscous solution.
3) 1-MID (80 mL) was added and one, two or three anhydrides selected from acetic—85 mL, propionic—132 mL and butyric 170 mL.
4) After 4 h incubation the excess anhydride was decomposed by addition of 3 L of water and the reaction mixture was poured into 2 vols of ethanol.
5) The precipitated Acylated Starch product was washed with ethanol (80% v/v) several times to remove the DMSO and other reactants and dried at 40° C. in a hot air room.
6) A control starch went through a sham procedure in which no 1-MID or anhydride was added to the starch.
7) The starches were ground to a fine powder and analysed.

Results

The DS of each of the Acylated Starch products produced above were similar. It will be understood that where only one anhydride is used at step 3, the acylated starch contains only 1 species of short chain fatty acid moiety. Where 2 anhydrides are used (for example, acetic acid and butyric acid), the acylated starch will contain a mixture of both acetic and butyric ester moieties. Where 3 anhydrides are used, the acylated starch will contain a mixture of all three fatty acid moieties.

### Example 3: Preparation of SCFA Enriched Diet

Large amounts (˜500 g) of acylated starch, were prepared by the large scale procedure detailed in Example 2 above.

A combination diet containing both acetylated and butyrylated starch was prepared and contained the following ingredients:
casein (200 g/kg)
methionine (1.5 g/kg)
sucrose (50 g/kg)
starch (251.5 g/kg)
corn oil (100 g/kg)
mineral mix (35 g/kg)
vitamin mix (10 g/kg)
choline tartrate (2 g/kg)
cellulose (50 g/kg)
acetylated starch (150 g/kg)
butyrylated starch (150 g/kg)

The percentage of acylated starch in the above diet is 30% (15%:15% acetylated:butyrylated starch).

An alternative combination diet can be prepared containing:

components as g/kg

| Ingredient | 15%/15% |
|---|---|
| Maize starch-3401C | 229.5 |
| Acetylated starch | 150 |
| Butyrylated starch | 150 |
| Casein | 200 |
| Sucrose | 100 |
| Sunflower Seed Oil | 70 |
| alpha cellulose | 50 |
| Mineral Mix AIN 93G | 35 |
| Vitamin Mix AIN 93VX | 10 |
| L-Cystine | 3 |
| Choline bitartrate | 2.5 |
| Total | 1000 |

The diets can be cold extruded into pellets, dried and stored at low temperature prior to use. Alternatively, the diets can be prepared as powders, for inclusion into foods as described herein.

It will be appreciated that a similar diet can be prepared using 300 g of an acylated starch containing both acetate and butyrate moieties (rather than 150 g of a starch acylated with only one species of short chain fatty acid).

### Example 4: Food Product Containing Acetylated and Butyrylated Starches

The acylated starches described herein can be used in powder form as supplements in various foods. For example:
A. Recipe for rapid dough containing acetylated and butyrylated starch:
   80 parts flour
   10 parts acetylated starch
   10 parts butyrylated starch
   2 parts fat
   2 parts salt
   1 part improver
   2.5 parts yeast
B. Other Foods that can be Supplemented Using the Acylated Starches Described Herein:
   The powders of acylated starches described above can also be added to a range of hot foods including pasta sauces, risotto, gravy, soups and porridge or to cold foods including milk, cereal, chocolate/vanilla custard, pudding and juices.

It will be understood that the invention disclosed and defined in this specification extends to all alternative combinations of two or more of the individual features mentioned or evident from the text or drawings. All of these different combinations constitute various alternative aspects of the invention.

1. A method for treating a condition associated with pathogenic immune cells, the method comprising or consisting of:
reducing the activity of, or number of, pathogenic immune cells in an individual in need thereof; and
providing in the individual, a therapeutically effective amount of at least one type of short chain fatty acid, ester or salt thereof
thereby treating the condition associated pathogenic immune cells.

2. The method of claim 1, wherein the condition is associated with effector and/or memory T cells, dysfunctional/ineffective regulatory T cell function or autoreactive T effector cells.

3. (canceled)

4. (canceled)

5. The method of claim 1, wherein the condition is an autoimmune disease.

6-12. (canceled)

13. The method of claim 1, wherein the method comprises reducing or treating inflammation in the individual.

14-16. (canceled)

17. The method of claim 1, wherein the method comprises reducing the abundance or presence of one or more autoantibodies in the individual.

18. The method of claim 1, wherein the condition is selected from the group consisting of: type 1 diabetes, psoriasis, rheumatoid arthritis, inflammatory bowel disease, coeliac disease, autoimmune hepatitis, myocarditis, lupus nephritis, systemic lupus erythematosus (SLE), non-alcoholic steatohepatitis (NASH), non-alcoholic fatty liver disease (NAFLD), multiple sclerosis, graft versus host disease or primary biliary cirrhosis.

19-22. (canceled)

23. The method of claim 1, wherein the reduction in the activity of pathogenic immune cells is a reduction in the activity of effector and/or memory T cells, or a reduction in the activity of Th1, Th17 and/or Th22 cells, or a reduction in the number of effector and/or memory T cells, or a reduction in the number of Th1, Th17 and/or Th22 cells.

24-29. (canceled)

30. The method of claim 1, wherein the step of reducing the activity of, or number of, pathogenic immune cells in an individual comprises or consists of administering to the individual an agent capable of reducing the viability of a T cell, or capable of depleting a T cell or population of T cells when administered to the individual.

31-34. (canceled)

35. The method of claim 1, wherein reducing the activity or number of pathogenic immune cells in the individual comprises administering an antibody that binds to an immune cell and results in a reduction in the viability of the immune cell.

36. The method of claim 1, wherein the short chain fatty acid, ester or salt thereof is provided to the individual after the activity of, or number of, pathogenic immune cells has been reduced in the individual.

37. The method of claim 1, wherein the short chain fatty acid, ester or salt thereof is provided to the individual at the same time as reducing the number of pathogenic immune cells in the individual.

38. (canceled)

39. (canceled)

40. The method of claim 1, wherein the at least one short chain fatty acid, ester or salt thereof, includes a combination of at least acetic acid and butyric acid, or esters or salts thereof.

41-47. (canceled)

48. The method of claim 1 wherein the short chain fatty acids are provided in the large intestine of the individual.

49. The method of claim 1 wherein the short chain fatty acid is provided in the colon of the individual.

50. (canceled)

51. The method of claim 1 wherein the short chain fatty acid is provided in the individual by oral administration to the individual of a dietary agent or pharmaceutical composition including said short chain fatty acid(s).

52. The method of claim 1, wherein the short chain fatty acid is provided in the individual by oral administration to the individual of a dietary agent, wherein the dietary agent includes a carrier molecule covalently bonded to at least one short chain fatty acid, wherein the covalent bond is resistant to degradation in the small intestine of the individual but is hydrolysable in the colon to provide free fatty acid in the colon of the individual.

53. The method of claim 52 wherein the carrier is a starch.

54-62. (canceled)

63. The method of claim 1 wherein the method comprises:
administering to the individual an agent that reduces the activity of, or number of, pathogenic immune cells in the individual;
administering to the individual a dosage form containing a therapeutically effective amount of a colonic composition, said composition consisting of:
a core consisting of a combination of at least two species of short chain fatty acid, wherein the short chain fatty acids are selected from acetic acid, butyric acid and propionic acid, or a pharmaceutically acceptable salt or ester thereof; and
at least one digestion-resistant layer covering said core,
said digestion-resistant layer disintegrating in the colon; and
releasing the core within the lumen of the colon.

64. (canceled)

65. (canceled)

66. The method of claim 1, wherein reducing the activity of, or number of, pathogenic immune cells in an individual in need thereof, comprises administering an antibody, or antigen binding fragment thereof, for binding to CD3, CXCR3 or CCR6.

67. The method of claim 66, wherein reducing the activity of, or number of, pathogenic immune cells in an individual in need thereof, comprises administering an antibody, or antigen binding fragment thereof, wherein the antibody or antigen-binding fragment thereof is a CD3-, CXCR3- or CCR-depleting antibody or antigen-binding fragment thereof.


SEQUENCE LISTING (3 sequences)

SEQ ID NO: 1 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 330 aa, Homo sapiens; identical to SEQ ID NO: 1

SEQ ID NO: 3 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK